(19) United States
(12) Patent Application Publication
(10) Pub. No.: US 2026/0013778 A1
(43) Pub. Date: Jan. 15, 2026

(54) Methods and Systems for Detecting Stroke

(71) Applicant: The Regents of the University of California, Oakland, CA (US)

(72) Inventor: Mauro Caffarelli, San Francisco, CA (US)

(21) Appl. No.: 18/993,797

(22) PCT Filed: Aug. 10, 2023
(86) PCT No.: PCT/US2023/029999

§ 371 Date: Jan. 13, 2025

Related U.S. Application Data
(60) Provisional application No. 63/371,328, filed on Aug. 12, 2022.

(51) Int. Cl. A61B 5/372; A61B 5/00; A61B 5/291; A61B 5/384

(52) U.S. Cl. CPC A61B 5/372; A61B 5/291; A61B 5/384; A61B 5/7275; A61B 5/7285

(57) ABSTRACT

Provided are methods of assessing whether a subject had a stroke and treating the subject accordingly. The methods include recording electroencephalography (EEG) data from electrodes positioned on the head of the subject. Afterwards, the electrical activity from a certain location on the subject's head is compared to the electrical activity at its contralateral location and to electrical activity measured at all electrodes. These relative electrical activities are used to determine the probability that a stroke occurred in a particular part of the brain, and the subject is treated accordingly.

### INTRODUCTION

Stroke is a devastating disease associated with significant neurological disability and healthcare costs.^{1,2} Early identification is critical for timely treatment with thrombolysis or mechanical thrombectomy, however the diagnosis is often delayed or misdiagnosed as a stroke mimic.³⁻⁵ The development of ancillary techniques for frontline providers to detect stroke is an important direction for patient care.^{1,6}

Quantitative electroencephalography (QEEG) has the potential to be a useful tool in the detection of cerebral ischemia.⁷ Brain tissue that is receiving inadequate perfusion will develop electrical suppression before reaching metabolic failure causing a stroke.^{8,9} These changes manifest within seconds of a loss in cerebral perfusion,^{10,11} suggesting that EEG could be used to create an early alarm system for stroke detection and prevention.

Although such a neurologist can be present at a large or specialized hospital, it is uncommon for such a neurologist to be immediately available when a patient has a stroke. For instance, if a citizen at their home or workplace believes they are having a stroke, they can contact emergency services, who will dispatch a paramedic to the citizen. However, paramedics commonly lack the training and knowledge to interpret EEG data for stroke detection. Thus, the paramedics must make a stroke assessment without the aid of EEG data. In addition, even after transportation to a hospital, a neurologist will not necessarily be available at the hospital.

In other cases, the patient experiences a stroke while already at a hospital. However, if the patient is unconscious (e.g. sleeping or in a coma), the patient cannot alert medical staff that they feel unwell. Thus, the medical staff will be unable to perform a stroke assessment or unaware that they should perform a stroke assessment. In addition, having a neurologist continually monitoring the EEG of a patient in the hospital for the possibility of stroke occurring would consume considerable resources.

### REFERENCES

1. Ferriero D M, Fullerton H J, Bernard T J, et al. Management of Stroke in Neonates and Children: A Scientific Statement From the American Heart Association/American Stroke Association. Stroke. 2019; 50(3). doi:10.1161/STR.0000000000000183
2. Gardner M A, Hills N K, Sidney S, Johnston S C, Fullerton H J. The 5-year direct medical cost of neonatal and childhood stroke in a population-based cohort. Neurology. 2010; 74(5):372-378. doi:10.1212/WNL.0b013e3181cbcd48
3. Mackay M T, Monagle P, Babl F E. Improving diagnosis of childhood arterial ischaemic stroke. Expert Review of Neurotherapeutics. 2017; 17(12):1157-1165. doi:10.1080/14737175.2017.1395699
4. Mackay M T, Churilov L, Donnan G A, Babl F E, Monagle P. Performance of bedside stroke recognition tools in discriminating childhood stroke from mimics. Neurology. 2016; 86(23):2154-2161.
5. Braun K P J, Kappelle L J, Kirkham F J, Frcpch M B. Diagnostic pitfalls in paediatric ischaemic stroke. Developmental Medicine & Child Neurology. 48(12):985-990. doi:10.1017/S0012162206002167.
6. Werho D K, Pasquali S K, Yu S, et al. Epidemiology of Stroke in Pediatric Cardiac Surgical Patients Supported With Extracorporeal Membrane Oxygenation. The Annals of Thoracic Surgery. 2015; 100(5):1751-1757. doi:10.1016/j.athoracsur.2015.06.020
7. Rosenthal E S, Biswal S, Zafar S F, et al. Continuous electroencephalography predicts delayed cerebral ischemia after subarachnoid hemorrhage: A prospective study of diagnostic accuracy: EEG Accurately Predicts DCI. Ann Neurol. 2018; 83(5):958-969. doi:10.1002/ana.25232
8. Foreman B, Claassen J. Quantitative EEG for the detection of brain ischemia. Critical Care. 2012; 2012(16:216). doi:10.1007/978-3-642-25716-2
9. Appavu B L, Temkit M H, Foldes S T, et al. Quantitative Electroencephalography After Pediatric Anterior Circulation Stroke. Journal of Clinical Neurophysiology. 2020; Publish Ahead of Print. doi:10.1097/WNP.0000000000000813
10. Kamitaki B K, Tu B, Wong S, Mendiratta A, Choi H. Quantitative EEG Changes Correlate With Post-Clamp Ischemia During Carotid Endarterectomy. Journal of Clinical Neurophysiology. 2021; 38(3):213-220. doi:10.1097/WNP.0000000000000686
11. Blume W T, Ferguson G G, McNeill D K. Significance of EEG changes at carotid endarterectomy. Stroke. 1986; 17(5):891-897. doi:10.1161/01.STR.17.5.891

### SUMMARY

Provided are methods of assessing whether a subject had a stroke and treating the subject accordingly. The methods include recording electroencephalography (EEG) data from electrodes positioned on the head of the subject. Afterwards, the electrical activity from a certain location on the subject's head is compared to the electrical activity at its contralateral location and to electrical activity measured at each electrode. These relative electrical activities are used to determine the probability that a stroke occurred in a particular part of the brain, and the subject is treated accordingly. Such methods can improve the accuracy and speed of stroke detection.

Notably, the present methods have the advantage of assessing stroke without the need for historical EEG data from the same patient. For example, in some cases the patient has symptoms of a stroke and paramedics are dispatched to the patient's home to assess the possibility of a stroke. Therefore, no historical EEG data exists and the paramedic cannot compare the patient's current electrical activity to normal, historical EEG activity from the same patient. However, the current methods employ a comparison between contralateral locations of the patient's brain, thereby allowing the detection of unsymmetrical activity. Thus, the current methods have the advantage of not requiring historical EEG data.

The comparison between contralateral brain locations provides other benefits as well. For example, a person without stroke undergoing anesthesia may have suppressed EEG activity that is “abnormal” compared to that of a healthy awake person. Despite having suppressed electrical brain activity, persons under anesthesia must still be assessed for the presence of stroke. In this way, comparing one side of the of the brain to the other confers the ability to detect stroke that is not dependent on comparison of the subject's EEG to “normative values,” that is, the aggregate of EEG data collected from healthy volunteers. Comparing the left-front brain to the right-front brain will show an asymmetry without the need for normative values, and therefore the stroke will be detected.

### BRIEF DESCRIPTION OF THE DRAWINGS

FIG. 1A shows raw EEG tracing used to calculate COIN for power bands between 4-16 Hz on all channels. Before and after refer to before and after stroke.

FIG. 1B shows values from each channel are averaged and mapped to the topographic visualizer. The “after” section shows dark blue on columns Fp1-F7 and Fp1-F3 (i.e. COIN values of −10) and light red in columns FB-T8 and C4-P4 (i.e. COIN values of +5).

FIG. 1C shows negative values from the topographic visualizer and summated to generate a summary value. The L side has dark blue (i.e. −4) COIN values and the R side has light red (i.e. +2).

FIG. 1D shows that a summary value is calculated every 4 seconds and smoothed using a 5-minute moving average (orange line).

FIG. 2 shows topographic visualization of COIN in order of increasing relative infarct volume (RIV). The first row shows neuroimaging, second row shows topographic visualization using full montage, third row shows topographic visualization using a limited circumferential montage. Circles on the first row indicate neuroimaging obtained before EEG, and stars on the first row indicate a seizure noted on the EEG. The darkest regions are dark blue, corresponding to very negative COIN values.

FIG. 3A shows summary of COIN values for all control subjects.

FIG. 3B shows summary of COIN values for all stroke subjects.

FIG. 3C shows summary COIN values with boxplots showing median and quartile ranges for control subjects in the first 6 hours of recording.

FIG. 3D shows summary COIN values with boxplots showing median and quartile ranges for stroke subjects against relative infarct volume in the first 6 hours of recording.

FIG. 4A shows average and standard error of the median values from each study comparing controls to all stroke patients, patients with strokes in anterior circulation, posterior circulation, relative infarct volume >5% and relative infarct volume >10%.

FIG. 4B shows receiver-operator characteristic curves from logistic regression.

FIG. 4C shows cutoff values determined using the Youden J statistic (sensitivity+specificity−1), with optimal cutoff values calculated as COIN value with the maximal Youden J Statistic.

FIG. 5A shows time independent distribution of COIN values.

FIG. 5B shows all strokes versus control for accuracy and AUROC.

FIG. 5C shows all strokes versus controls for specificity and sensitivity.

FIG. 5D shows RIV >5% versus controls for accuracy and AUROC.

FIG. 5E shows RIV >5% versus controls for specificity and sensitivity.

FIG. 6A shows COIN values as a function of stroke volume.

FIG. 6B shows sensitivity as a function of 1−specificity for the FIG. 6A data.

FIG. 6C shows sensitivity, specificity, and sensitivity+specificity−1 for the FIG. 6A data.

FIG. 7A shows a three-dimensional graph where qᵢⱼ is a function of rᵢⱼ and sᵢⱼ. The upper regions are red and the lower regions are blue. The middle regions are gray.

FIG. 7B shows a three-dimensional graph where qᵢⱼ is a function of rᵢⱼ and sᵢⱼ wherein qᵢⱼ is set to zero when rᵢⱼ multiplied by sᵢⱼ is less than zero.

FIG. 8 shows a flow diagram of 694 inpatient EEG encounters being sorted into different populations.

FIG. 9A shows a topographical visualization of COIN in a patient during a first time period, along with the raw EEG data. The bottom axis ranges from 0 seconds to 4 seconds. The vertical axis shows the data from 16 different channels. The topographical visualization shows very faint blue in the front-right brain of the patient. Notably, the right side of the patient is noted by “R” and is shown on the left of the figure.

FIG. 9B shows data corresponding to the FIG. 9A patient at a second time period. A deep blue region developed in the rear-right side of the patient's brain. Dark region is dark blue and has very negative COIN values.

FIG. 9C shows data corresponding to the patient of FIGS. 9A and 9B at a third time period. A very deep blue region developed in the rear-right part of the brain, and a very red region developed on the left side of the brain. R side region is dark blue and has very negative COIN values.

FIG. 10 shows COIN neuroimages of patient brains, stroke volume, C values, and topographical COIN visualization in order of ascending stroke volume. L: Left, R: Right, ACA: Anterior Cerebral Artery, MCA: Middle Cerebral Artery, PCA: Posterior Cerebral Artery NS: not specified, Vol: Stroke Volume. The largest and darkest regions are dark blue and have very negative COIN values. The contralateral locations are light red and have slightly positive COIN values.

### DETAILED DESCRIPTION

Provided are methods of assessing whether a subject had a stroke and treating the subject accordingly. The methods include recording electroencephalography (EEG) data from electrodes positioned on the head of the subject. Afterwards, the electrical activity from a certain location on the subject's head is compared to the electrical activity at its contralateral location and to electrical activity measured at each electrode. These relative electrical activities are used to determine the probability that a stroke occurred in a particular part of the brain, and the subject is treated accordingly.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The terms “subject” and “patient” are used interchangeably herein to refer to an animal, such as a human.

The terms “determining”, “measuring”, and “assessing” are used interchangeably herein.

### METHODS

Provided are methods of assessing whether a subject had a stroke and treating the subject accordingly. In some instances, the methods include:
(a) recording electroencephalography (EEG) data from electrodes positioned at scalp locations on the head of the subject;
(b) determining relative electrical activities from the EEG data, wherein the determining comprises:
   determining the relative electrical activity at each scalp location compared to electrical activity at all scalp locations; and
   determining the relative electrical activity at each scalp location compared to electrical activity at its corresponding contralateral scalp location;
(c) determining a high probability of stroke based on the determined relative electrical activities; and
(d) treating the subject for a high probability of stroke.

### Recording EEG Data

As described above, the exemplary methods include recording electroencephalography (EEG) data by measuring electrical activity with electrodes positioned at scalp locations on the head of the subject. The term “electrogram” (EGM) is used herein to refer to the recording of the electrical activity. Although the electrodes are positioned at the scalp of the subject, the recorded EEG data corresponds to the activity of the brain underneath such scalp locations.

In some cases, step a) of recording EEG data comprises the sub-steps of i) recording electrical activity from electrodes and ii) generating EEG data from the recorded electrical activity.

As such, electrical activity is recorded by actual electrodes on the subject's head. However, the EEG data can be classified by its “channel”, wherein there is one channel for each scalp location.

In some cases, a channel of EEG data is generated from the electrical activity recorded by a single electrode. For example, there can be four electrodes positioned at four scalp locations. These four electrodes record four sets of electrical activity, and the four sets of electrical activity are the four channels of EEG data corresponding to the four scalp locations. For example, there can be “Fp1” electrode and a “F7” electrode.

In other cases, a channel of EEG data is generated from the electrical activity recorded by a first electrode, the electrical activity recorded by a second electrode, and optionally electrical activity recorded by additional electrodes. Typically the first and second electrodes are positioned close to each other, e.g. less than 5 cm apart, such as less than 4 cm, less than 3 cm, less than 2 cm, less than 1 cm, or less than 0.5 cm. By using two (or more) electrodes to create a certain channel, the signals from adjacent electrodes can be used to cancel out any distant physiologic electrical activity, e.g. from the heart. For example, the electrical activity recorded by the first electrode can be subtracted from the electrical activity recorded by the second electrode. As such, the brain's electrical activity near a certain scalp location can be estimated by recording electrical activity by two electrodes near or at the scalp location, and then combining the two recordings to generate a particular channel of EEG data corresponding to the particular scalp location. If such electrodes are named “Fp1” and “F7”, then the corresponding channel can be named “Fp1-F7”.

At least one pair of scalp locations are located “contralateral” to each other. The patient's head can be referred to as being bisected by a medial plane that separates the right side of the subject's head from the left side. The term “contralateral” is used herein to refer to a scalp location that is on the opposite side of the medial plane from another scalp location. Stated in another manner, if a first scalp location is contralateral to a second scalp location, then reflecting the first scalp location through the medial plane will arrive at the second scalp location. The term “medial plane” is used interchangeably with “median plane” and “mid-saggital plane”.

In some cases there are four or more scalp locations, e.g. including two pairs of contralateral scalp locations. In some embodiments, there are six or more scalp locations, e.g. including three pairs of contralateral scalp locations. In some cases, there are 8 or more scalp locations and 4 or more pairs, or 10 or more scalp locations and 5 or more pairs. In some cases, the EEG data channel is generated from a single electrode at the scalp location. In some cases, the EEG data channel is generated from two or more electrodes at or near the scalp location.

For simplicity, the embodiments below are sometimes described wherein a single electrode's recordings are used to generate the EEG data channel for a particular scalp location. However, it is understood that each embodiment could be modified so that the EEG data channel for a particular scalp location is derived from multiple electrodes.

In some cases, the recording of EEG data is performed for 1 second to 25 seconds, such as 2 seconds to 10 seconds. In some cases, the EEG data is recorded for 4 seconds. As such, the recordings for these time periods is used to determine the referential and symmetrical electrical activity.

### Determining Referential Electrical Activity

The exemplary method includes determining the relative electrical activity at each scalp location compared to electrical activity at all scalp locations, which is also referred to herein as determining “referential electrical activity” or “reference electrical activity”. For example, if four total electrodes are present, the electrogram from the first electrode can be compared to electrograms from the second, third, and fourth electrodes. In addition, the electrogram from the electrode 2 can be compared to electrograms from electrodes 1, 3, and 4; the electrogram from electrode 3 can be compared to electrograms from electrodes 1, 2, and 4; and the electrogram from electrode 4 can be compared to electrograms from electrodes 1, 2, and 3.

In some cases, such determining of referential electrical activity involves determining the mean (i.e. the average) of electrical activity from all electrodes. As such, the referential electrical activity of the first electrode involves comparing the raw electrical activity of the first electrode to the mean electrical activity of all electrodes.

### Determining Symmetry Electrical Activity

In addition, the exemplary method includes determining the relative electrical activity at each scalp location compared to electrical activity at its corresponding contralateral scalp location, which is also referred to herein as determining “symmetry electrical activity”. For instance, in an exemplary embodiment, electrode 1 is contralateral to electrode 2 and electrode 3 is contralateral to electrode 4. Determining the symmetry electrical activity of electrode 1 involves comparing the raw electrical activity at electrode 1 to raw electrical activity at electrode 2, which is its contralateral electrode. Similarly, the symmetry electrical activity of electrode 2 is determined by electrode 2 activity compared to electrode 1 activity. In addition, symmetry electrical activity of electrode 3 is determined by comparing electrode 3 and electrode 4, and symmetry electrical activity of electrode 4 is determined by comparing electrode 3 and electrode 4 activity.

In some embodiments, determining the symmetry electrical activity at an electrode includes dividing the electrical activity at the electrode by the electrical activity of its contralateral electrode.

### Determining the Probability of a Stroke

The exemplary method includes determining the probability that the subject experienced a stroke based on the determined relative electrical activities. Stated in another manner, the method includes determining the probability that the subject experienced a stroke based on the determined referential electrical activity and the determined symmetry electrical activity.

As described above, the method involves determining referential electrical activity for at least one scalp location. Similarly, the method involves determining symmetry electrical activity for at least one scalp location. As such, the probability of stroke can be determined by considering one scalp location at a time. For instance, the referential activity at electrode 1 can be compared to the symmetry activity at electrode 1 in order to determine the probability of a stroke at the location of electrode 1. In some embodiments, the determination of stroke probability is repeated for at least one other scalp location (e.g. electrode 2), e.g. for all scalp locations. Thus, determining the probability of a stroke can include determining the probability of a stroke at each scalp location corresponding to each electrode. Thus, the method can not only determine probability of stroke, but the method can also advantageously determine which region of the brain is most likely to have experienced a stroke.

In some cases, the determination is determination of an ischemic stroke, whereby blockage of a blood vessel in the brain or neck causes transient or permanent cessation of blood flow to a portion of the brain. In some cases, the determination is determination of a hemorrhagic stroke, whereby rupture of a blood vessel in the brain or skull causes bleeding to occur in the brain or skull. The ischemic or hemorrhagic stroke could be spontaneous or occur from any cause, including but not limited to: traumatic brain injury, embolus (whereby a material such as blood clot, air, fat, or foreign body travels through the blood stream to the brain, where it becomes lodged inside a blood vessel), atherosclerotic disease, brain aneurysm, or inflammatory disease (cancer, autoimmune, or infection). In some cases, the determination is determination of any disease to an area of the brain that might cause differences in electrical activity, such as a brain tumor or infection (such as infectious abscess)

### Treating the Subject for a High Probability of Stroke

In cases wherein the determined probability of stroke is high, the exemplary method further includes treating the subject for a high probability of stroke. By determining the risk of stroke, the earlier steps in the method provide the advantage of a more accurate diagnosis, thereby aiding in the triage and treatment of possible stroke patients. In some embodiments, a high probability of stroke means that the estimated probability of stroke is 1% or more, such as 5% or more, 10% or more, 25% or more, or 50% or more. In some embodiments, a high probability of stroke means that a healthcare professional determines that the subject should be treated for stroke, e.g. by conducting additional stroke detection measurements or by a medical procedure to minimize or reverse the effects of the possible stroke.

In some cases, treating for high probability of stroke includes performing additional stroke detection measurements, e.g. a brain magnetic resonance image (MRI) or a head computed tomograph (CT) scan. For example, such measurements can be conducted in a hospital.

In some cases, such treatment includes a medical procedure to minimize or reverse the effects of the possible stroke. In some cases, such treatment includes medical or surgical intervention to reverse or minimize the effect of the probable stroke. For example, the brain surgery can be performed in order to inhibit bleeding during a hemorrhagic stroke or to correct a lack of blood flow in an ischemic stroke.

### Before Recording EEG Data

Before recording the EEG data, in some cases the subject had an elevated risk of stroke. In other words, the method is performed because the subject had an elevated risk of stroke. For example, in some cases the elevated risk of stroke is selected from the group consisting of: altered mental status, loss of motor function, loss of sense of touch in a body part, dizziness, headache, and difficulty speaking. In some embodiments, the elevated risk of stroke is selected from the group consisting of: head trauma, hematoma (e.g. epidural, subdural), and subarachnoid hemorrhage. In some cases, the elevated risk of stroke is selected from the group consisting of: currently receiving surgery (e.g. on the brain, heart, or blood vessels) and receiving surgery within the past 30 days.

In some embodiments, the subject is located in a medical facility (e.g. hospital) at the beginning of the recording of the EEG data. For instance, the subject could being receiving surgery or the subject could be recovering after surgery. In some embodiments, the subject was recently brought to the medical facility, e.g. within the last 24 hours, because of an elevated risk of stroke, e.g. head trauma or dizziness.

In some cases, the subject is located outside a medical facility at the beginning of the recording of the EEG data. For instance, a medical professional (e.g. a paramedic) could be dispatched to contact and assess a patient having symptoms consistent with stroke, and the method can help increase the accuracy of diagnosis. In such cases, the step of treating the subject for a high probability of stroke can include transporting the subject to a medical facility, notifying a medical facility of the high probability that the subject experienced a stroke, or a combination thereof.

### Repeating if High Probability is not Detected Upon an Initial Assessment

In some cases, the method includes recording EEG data, determining relative electrical activities from the EEG data, and determining the probability of a stroke. If the determined probability of stroke is high, then the method includes treating the subject for the high probability of stroke.

However, if the determined probability of stroke is not high (e.g. the probability is low), then the method can include repeating the recording step and the two determining steps. Stated in another manner, EEG data can be continuously recorded and the two determining steps (labelled as b and c above) can be continuously repeated accordingly. Thus, the method can also be referred to as a method of continuously monitoring a subject for a stroke. The continuous monitoring can be performed for 1 minute or more, such as 10 minutes or more, 30 minutes or more, 1 hour or more, or 4 hours or more. In addition, the recording and two determining steps can be repeated with a frequency of at least once per hour, such as at least once per 10 minutes, at least once per minute, at least once per 10 seconds, or at least once per second.

Determining Electrical Activity with Different Time Periods

In some cases, the probability of stroke is evaluated at each different time point independently. For example, data can be recorded for a single time period (e.g. four seconds) and this single time period can be used to determine probability of stroke. This situation can be referred to as a first time period and a second time period.

In other cases, the probability of stroke is evaluated by considering readings from multiple consecutive time periods. For instance, data can be recorded for a first time period (e.g. four seconds) and then data can recorded for a second time period (e.g. another four seconds). In such cases, the determination of stroke is based on a combination of data from the first time period and second time period. Such combinations can help “smooth” the data and reduce the effects of random error, e.g., by using a “moving average”. In some cases, each time period of recorded data ranges from 0.5 seconds to 15 seconds, and each stroke determination is made using data from between 2 time periods and 200 time periods. In some cases, the determination of stroke is made by averaging the r(f,j) and s(f,j) values from different time periods. In some cases, the determination of stroke is made by averaging g(f,j) values from different time periods. In some cases, the determination of stroke is made by averaging m(j) values from different time periods.

In some cases, the method further comprises:
repeating the recording of the EEG data during one or more additional time periods;
determining the relative electrical activities from the EEG data from one of the one or more additional time periods;
generating a combined relative electrical activity by combining the relative electrical activity from the EEG data from the first time period and the relative electrical activity from the EEG data from the one or more additional time periods; and
determining a high probability of stroke based on the combined relative electrical activities.

Such a method can help improve data quality by reducing the effect of random error or fluctuations in the readings.

For example, in some cases the one or more additional time periods consists of a second time period. As such, the combined relative electrical activity is based on a combination of the first and second time periods only. As another example, if there are 2 additional time periods, then the stroke probability is assessed by 3 total time periods. Thus, in some cases there are 1 or more additional time periods, such as 2 or more, 3 or more, 4 or more, 5 or more, 10 or more, 20 or more, 30 or more, or 50 or more. In some cases, each time period ranges from 1 second to 5 minutes, such as 2 seconds to 20 seconds.

In some embodiments, the combination of relative electrical activities from different time periods comprises averaging the relative electrical activities over time.

### Mathematical Aspects of Determining Relative Electrical Activities

In some cases, determining the relative electrical activities from the EEG data comprises generating a matrix from the EEG data. As used herein, the term “matrix” refers to an array of numbers.

In some embodiments, the method includes generating matrix A from the EEG data, wherein matrix A comprises elements a(t, j), wherein a(t, j) refers to the amplitude of electrical activity at time t and scalp location j. Stated in another manner, an electrode at a first scalp location (i.e. j=1) can record electrical activity, either in an analog or a digital format. If initially recorded in analog, the initial analog data is converted to a digital format. The EEG data can be recorded for any suitable period of time, e.g. from 1 second to 10 minutes. The EEG data can be recorded at any suitable frequency, e.g. from once every 10 milliseconds to once every 10 seconds.

As such, matrix A is a two-dimensional matrix, wherein the first dimension corresponds to the time that the data is recorded, the second dimension corresponds to the identity of the channel, e.g. a channel derived from a single electrode or multiple electrodes, and the value at a particular matrix location is the amplitude of electrical activity.

In some embodiments, the method further includes generating matrix B from matrix A, wherein matrix B comprises elements a(f, j), wherein a(f, j) refers to the power of electrical activity at frequency f and scalp location j. For example, applying a Fourier transform to matrix A (which recites data as a function of time) can be used to generate matrix B (which recites the intensity of electrical activity as a function of frequency). As such, converting matrix A to matrix B can be used to convert the raw data recorded from the channels into processed data showing the relative intensity of brain waves at different frequencies. For example, in some cases the frequencies of matrix B can range from 2 Hz to 18 Hz. In some cases, matrix B includes a row for frequencies at 1 Hz intervals, e.g. matrix B includes rows corresponding to 4 Hz, 5 Hz, 6 Hz, 7 Hz, 8 Hz, 9 Hz, 10 Hz, 11 Hz, 12 Hz, 13 Hz, 14 Hz, 15 Hz, and 16 Hz. For example, if EEG data was recorded from 4 channels and 12 frequencies ranges are used, then matrix B would have the size of a 4×12 matrix. In some cases, a single frequency range is used. In other cases, two or more frequency ranges are used, such as four or more, eight or more, or twelve or more.

In some embodiments, matrix B is used to generate referential matrix R and symmetry matrix S.

Referential matrix R includes elements r(f, j), wherein r(f, j) refers to the relative power of electrical activity at frequency f and scalp location j compared to the power of electrical activity at frequency f at all scalp locations. As described above, such determining of referential electrical activity can involve determining the mean (i.e. the average) of electrical activity from all channels. The mean of electrical activities from all channels can be described by the equation:

$$\frac{1}{M} {\sum }_{j = 1}^{M} a ( f , j )$$

wherein M is the total number of scalp locations j.

As described by the equation above, when different frequency ranges are used (e.g. 4 Hz, 5 Hz . . . 15 Hz, 16 Hz), the calculations are performed separately for the separate frequency ranges.

In some cases, determining each r(f, j) includes dividing each a(f, j) by the mean of all electrical activities. In some embodiments, each r(f, j) is determined according to the equation:

$$r ( f , j ) = {log}_{2} ( \frac{a ( f , j )}{\frac{1}{M} {\sum }_{j = 1}^{M} a ( f , j )} )$$

wherein M is the total number of scalp locations j.

Symmetry matrix S includes elements s(f, j), wherein s(f, j) refers to the relative power of electrical activity at frequency f and scalp location j compared to the power of electrical activity at frequency f and scalp location j* that is contralateral to scalp location j.

As describe above, in some cases determining s(f, j) includes dividing the electrical activity at location j by the electrical activity at j*, its contralateral location. In some cases, determining s(f, j) includes additional mathematical operations, such as determining s(f, j) according to the equation:

$$s ( f , j ) = {log}_{2} ( \frac{a ( f , j )}{a ( f , {j}^{*} )} )$$

Thus, in some embodiments of the method, determining the referential electrical activities and symmetrical electrical activities include the steps of: (i) generating matrix A from the EEG data, wherein matrix A describes amplitude over time; (ii) generating matrix B from matrix A, wherein matrix B describes power over one or more frequency ranges; (iii) generating referential matrix R by comparing electrical activity from a channel to all channels; and (iv) generating symmetry matrix S by comparing electrical activity from a channel to its contralateral channel.

### Mathematical Aspects of Determining Probability of Stroke

After generating the referential and symmetry electrical activities, the method includes determining the probability that the subject experienced a stroke.

In cases wherein matrices R and S were generated, determining the probability of stroke can include:
generating gating matrix G comprising elements g(f, j) by combining matrix R with matrix S;
generating map matrix M comprising elements m(j), wherein m(j) is generated by averaging g values for the same scalp location j over different frequencies f;
determining the probability of stroke based on map matrix M.

In some cases, each g(f, j) is calculated according to the equation:

$$g ( f , j ) = {r ( f , j )}^{2} \cdot s ( f , j )$$

In some cases, after calculating such g(f, j) values, the G matrix is further modified. For example, in some cases each g(f, j) value is changed to zero if its corresponding r(f, j) value has the opposite sign of its corresponding s(f, j) value. For example, for the 4 Hz frequency and channel 1, if r(4, 1) is negative but s(4, 1) is positive, then g(4, 1) is changed to zero. Similarly, if r(4, 1) is positive but s(4, 1) is negative, then g(4, 1) is changed to zero. In some cases, this manipulation avoids the generation of a false signal at a scalp location where the symmetry signal is low but the referential signal is high, and vice versa.

As described above, m(j) is generated by averaging g values for the same scalp location j over different frequencies f. This mathematical operation can also be represented by the equation:

$$m ( j ) = \frac{1}{F} {\sum }_{f - initial}^{f - final} g ( f , j )$$

wherein f is a variable representing each frequency range and F is the number of frequency ranges. For instance, if three frequency ranges of 4 Hz, 6 Hz, and 8 Hz were used, then then f-initial would be 4 Hz and f-final would be 8 Hz and F would be three.

In some cases, a high probability of stroke is determined when one or more m(j) values is within a predetermined threshold range, e.g. one or more m(j) values is less than −10, such as less than −15, less than −20, less than −25, less than −30, less than −35, or less than −40.

Additionally, value “C” is the sum of all negative m(j) values, as shown in the equation:

$$C = {\sum }_{j = 1}^{M} m ( j ) for all m ( j ) where m ( j ) < 0$$

wherein M is the total number of scalp locations j.

In some cases, a high probability of stroke is determined when the sum of all negative m(j) values (i.e. value C) is within a predetermined threshold range, e.g. less than −10, less than −15, less than −20, less than −25, less than −30, less than −35, or less than −40. In some cases, the controller is configured to detect if the size of the stroke is above 100 ml or below 100 ml with a sensitivity of 80% or more and a specificity of 85% or more.

### Size of Stroke

In some embodiments wherein a stroke is detected, the method further comprises determining the size of the stroke. For instance, a stroke can be considered large if the value of C is less than −20. In some cases, a large stroke corresponds to a volume of 100 ml or more, and small stroke corresponds to a volume of less than 100 ml. In some cases, the size of the stroke is determined to be 100 ml or more based on a C value of −20 or less, and the size of the stroke is determined to be less than 100 ml based on C value of greater than −20.

### Controllers

Aspects of the disclosure include a controller for assessing whether a subject had a stroke and communicating the results of the assessment. In some cases, the controller is configured to:
a) obtain electroencephalography (EEG) data from electrodes positioned at scalp locations on the head of the subject;
b) determine relative electrical activities from the EEG data, wherein the calculating comprises:
   determining the relative electrical activity at each scalp location compared to electrical activity at all scalp locations; and
   determining the relative electrical activity at each scalp location compared to electrical activity at its corresponding contralateral scalp location;
c) determine the probability that the subject experienced a stroke near one or more scalp locations based on the relative electrical activities; and
d) electronically instruct a communication device to communicate the determined probabilities.

The EEG data is recorded from three or more channels at three or more scalp locations, such as four or more channels at four or more scalp locations, such as six or more channels at six or more scalp locations, eight or more, ten or more, twelve or more, or fourteen or more. Each channels is positioned at a single scalp location. As described above, each channel of EEG data can be derived from a single electrode or multiple electrodes, but in each case a particular channel corresponds to a particular scalp location.

At least one pair of channels are placed “contralateral” to each other. The patient's head can be referred to as being bisected by a medial plane that separates the right side of the subject's head from the left side. The term “contralateral” is used herein to refer to a scalp location that is on the opposite side of the medial plane from another scalp location. Stated in another manner, if a first scalp location is contralateral to a second scalp location, then reflecting the first scalp location through the medial plane will arrive at the second scalp location. As such, in some cases there are two or more pairs of contralateral channels, such as three or more pairs, four or more pairs, five or more pairs, six or more pairs, or seven or more pairs.

Determining the relative electrical activities from the EEG data (i.e. step b) can be performed, in some cases, using the techniques and formulas described above regarding the methods. Similarly, determining the probability that the subject experienced a stroke near one or more scalp locations based on the relative electrical activities (i.e. step c) can also be performed, in some cases, using the techniques and formulas described above regarding the methods.

As described above, step d) of the procedure performed by the controller includes electronically instructing a communication device to communicate the determined probabilities.

In some cases, communicating the determined probabilities comprises providing a visual image indicating the one or more determined probabilities. For example, the visual image can be displayed by a light-emitting diode display, such as a computer monitor or a television.

In some embodiments, the image comprises a symbol representing a top view of the head of the subject. In some cases, the image uses different colors to show different relative probabilities that a stroke occurred near different scalp locations. In some embodiments, the different colors comprise a gradient between three or more colors. For instance, the gradient can be from blue to white to indicate a gradient of electrical underactivity (e.g. stroke) from high to low. In some cases, the gradient is from red to white to indicate a probability of electrical overactivity (e.g. seizure) from high to low.

In some cases, the controller is further configured to electronically instruct an alert device to provides a visual alert, an auditory alert, or a combination thereof when the determined probability near one or more scalp locations is within a predetermined threshold range. For example, the alert device could be a speaker that generates the auditory alert, such as a loud buzzing sound. In some cases, the alert device provides a visual alert, such as a flashing light. In some cases, the visual alert is provided by a visual notification on a screen, such as a computer monitor, e.g. wherein the visual notification repeatedly flashes in order to attract the attention of a user, such as a nurse. In some cases, the alert (visual, auditory, or both) is provided to a wearable device, such as a personal notification device (e.g. a pager) that can be worn by a nurse while traveling through a medical facility.

In some cases, the controller is configured to repeat steps a), b), c), and d). If the controller provides an alert, the controller can also repeatedly provide the alert. In some embodiments, the controller repeats the steps within 10 minutes or less, such as within 5 minutes or less, 2 minutes or less, 1 minute or less, or 10 seconds or less. As such, the controller can be referred to as “monitoring” for the possibility of a stroke by repeatedly performing steps a) through d) repeatedly.

The controller can be configured to detect an ischemic stroke, a hemorrhagic stroke, or a combination thereof. The controller can be configured to determine the size of the stroke, e.g. as discussed above regarding the methods by setting an alert based on a threshold value of C. As such, the controller can advantageously detect even relatively small strokes, e.g. of less than 100 ml, such as 25 ml to 100 ml by setting a threshold value of −10 for C. The controller can also be configured to detect only large strokes, e.g. of greater than 100 ml, such as 100 ml to 300 ml, by setting a threshold value of −30 for C.

The controller can achieve detection of stroke with high sensitivity and specificity. Such terms are discussed in the art, such as by Sheffler and Huecker (“Diagnostic Testing Accuracy: Sensitivity, Specificity, Predictive Values and Likelihood Ratios”) and Power et al (“Principles for high-quality, high-value testing”, BMJ Evidence-Based Medicine, doi: 10.1136/eb-2012-100645). Also discussed in the art is the term “accuracy”.

Such terms are described herein based on the equations:

$$Sensitivity = \frac{True Positives}{True Positives + False Negatives}$$

$$Specificity = \frac{True Negatives}{True Negatives + False Positives}$$

$$Accuracy = \frac{True Positives + True Negatives}{True Positive + True Negatives + False Positives + False Negatives}$$

The present controllers can advantageously detect strokes with both high sensitivity and high selectivity. Thus, the percentages of false positives and false negatives are relatively low. Increasing or decreasing the threshold value being used can favor either sensitivity or specificity. In some cases, the controller can detect stroke with a sensitivity of 50% or more and a specificity of 70% or more, e.g. using EEG data collected for 6 hours or less, such as 4 hours or less, 2 hours or less, 30 minutes or less, or 5 minutes or less. In some cases, the controller can detect stroke with a sensitivity of 70% or more and a specificity of 90% or more, e.g. using EEG data collected for 6 hours or less, such as 4 hours or less, 2 hours or less, 30 minutes or less, or 5 minutes or less. Such detections can be performed using EEG data collected for 6 hours or less, such as 4 hours or less, 2 hours or less, 30 minutes or less, or 5 minutes or less. For example, a predetermined threshold value of −15 or −20 for C can be used.

Some strokes are quite small and are therefore harder to detect. However, strokes that affect larger areas of the brain are more dangerous but also more readily detected. Thus, in some cases the controller can detect a stroke with an infarct volume of 5% or more with a sensitivity of 75% or more and a specificity of 80% or more. “Infarct volume” refers to the percentage of the brain that is affected by the stroke. In some cases the controller can detect a stroke with an infarct volume of 5% or more with a sensitivity of 90% or more and a specificity of 90% or more. Such detections can be performed using EEG data collected for 6 hours or less, such as 4 hours or less, 2 hours or less, 30 minutes or less, or 5 minutes or less. For example, a predetermined threshold value of −15 or −20 for C can be used.

Such sensitivity and specificities can in some cases be higher than those of a skilled artisan simply considering the raw EEG data itself.

### Systems

Also provided is a system for assessing whether a subject had a stroke and communicating the results of the assessment, wherein the system comprising:
a controller as described above; and
one or more of the communication device, an alert device, and the electrodes.

### Kits

Also provided is a kit for assessing whether a subject had a stroke and communicating the results of the assessment, wherein the system comprising:
a controller as described above; and
packaging containing the controller.

In some cases, the kit further comprises one or more of the communication device, an alert device, and the electrodes contained in the packaging.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used but some experimental errors and deviations should be accounted for.

### Example 1: Recognizing Ischemic Stroke in Children

### Overview

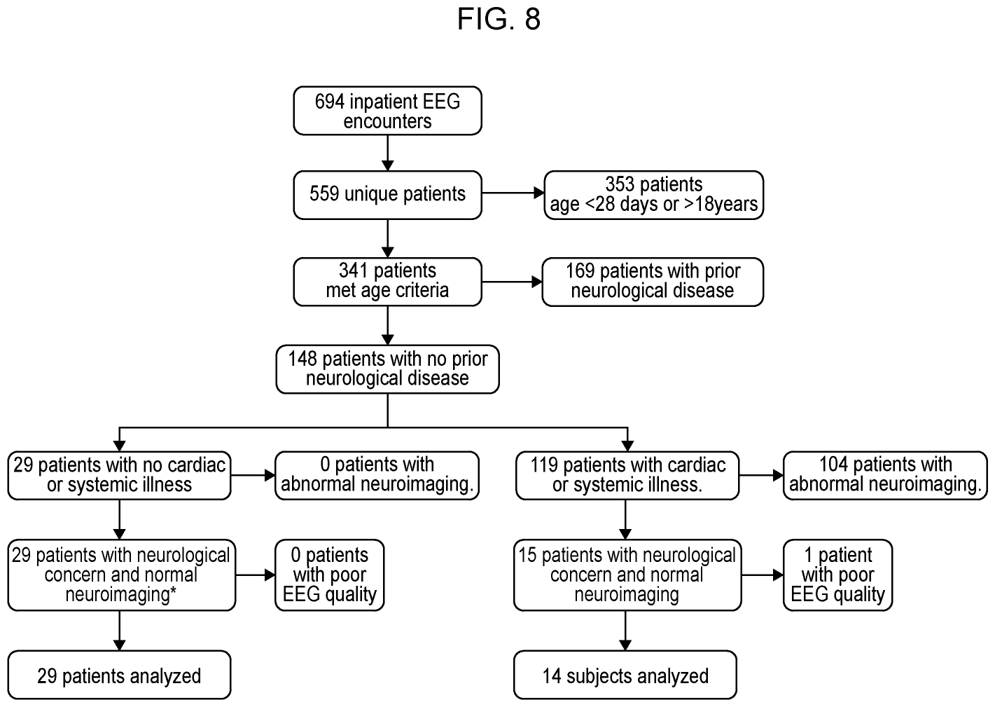

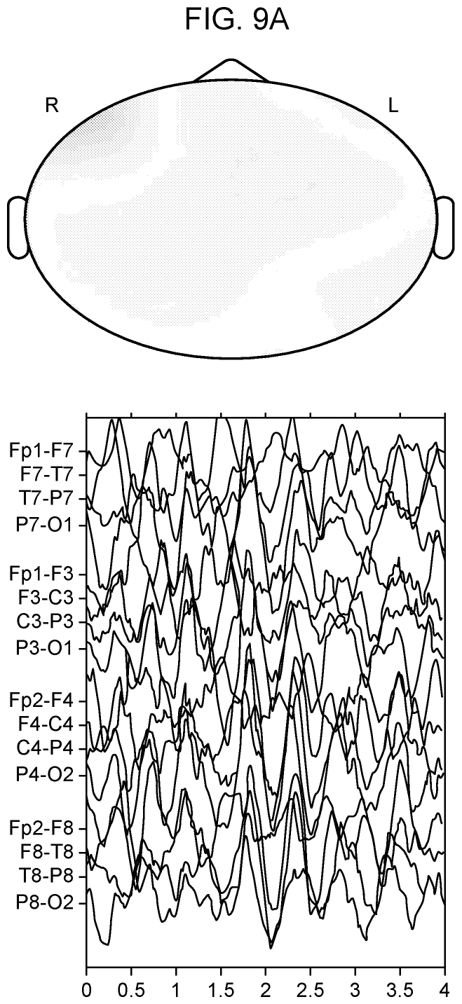

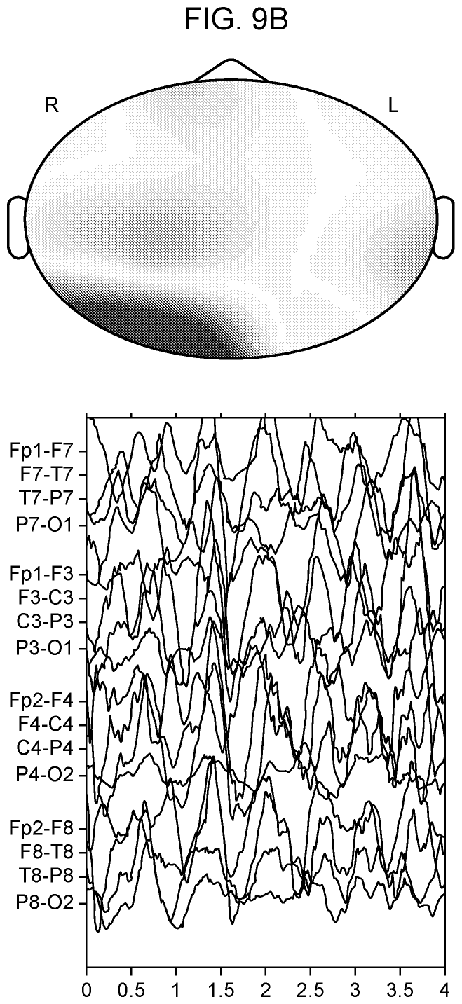

EEG data was recorded for three different child patient populations: patients with confirmed stroke, “control” patients who were not diagnosed with stroke and who were healthy, and “control” patients who were not diagnosed with stroke but who were sick with a certain other condition. FIG. 8 shows a flow diagram of 694 inpatient EEG encounters being sorted into different populations. The data was mathematically analyzed to generate COIN (Correlate of Injury to the Nervous System) which was used to determine the likelihood of ischemic stroke in the child. This study demonstrates that a continuous and quantitative measure of brain asymmetry derived from scalp EEG data has high sensitivity and specificity for detecting acute strokes with >5% relative infarct volume in hospitalized children.

### Study Population

Stroke subjects were children admitted to a pediatric hospital with confirmed stroke on neuroimaging, the control group had either normal imaging or a normal neurological exam. Both groups included children aged 28 days to 18 years. Subjects in the Seizures in Pediatric Stroke (SIPS II)¹³ database were sampled from multiple institutions and children in the matched control group were consecutively sampled from University of California San Francisco (UCSF) Benioff Children's Hospital (BCH) after applying selection criteria.

Inclusion criteria for both groups were initiation of EEG within 48 hours of stroke or hospital admission and a minimum continuous recording period of 6 hours. Control patients were excluded for premorbid diagnosis of epilepsy, neurological malignancy, prior stroke, neonatal hypoxic ischemic encephalopathy, or concurrent diagnosis of cardiac arrest, acute liver of kidney dysfunction, diabetic ketoacidosis, toxic ingestion, syndromic illness, or genetic abnormalities. Data on demographic variables, medication administration, qualitative EEG findings, and neuroimaging findings including relative infarct volume (RIV, calculated as infarct volume over total brain volume) on the stroke patients were extracted from SIPS II case report forms.

### EEG Processing

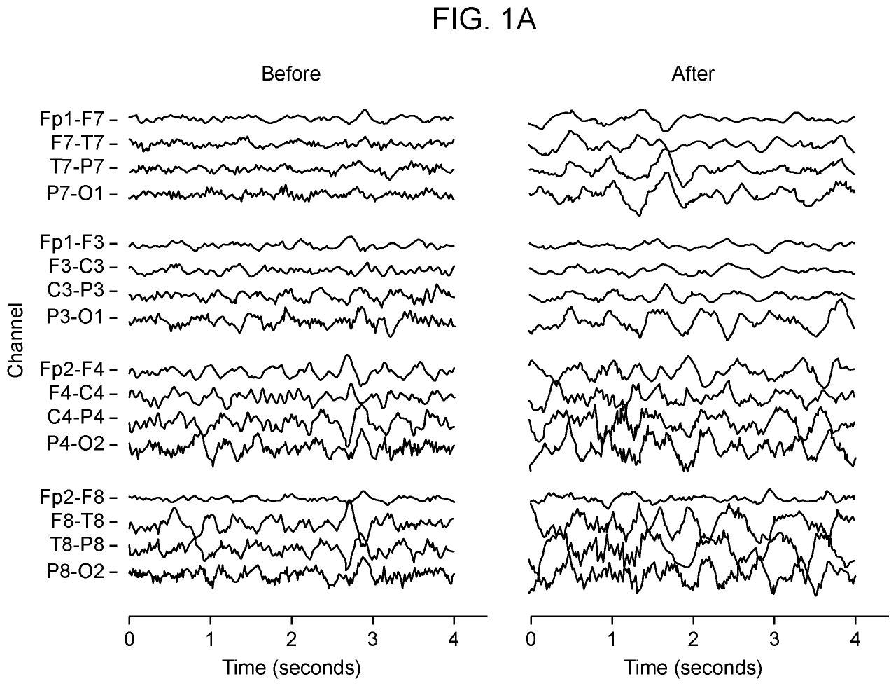

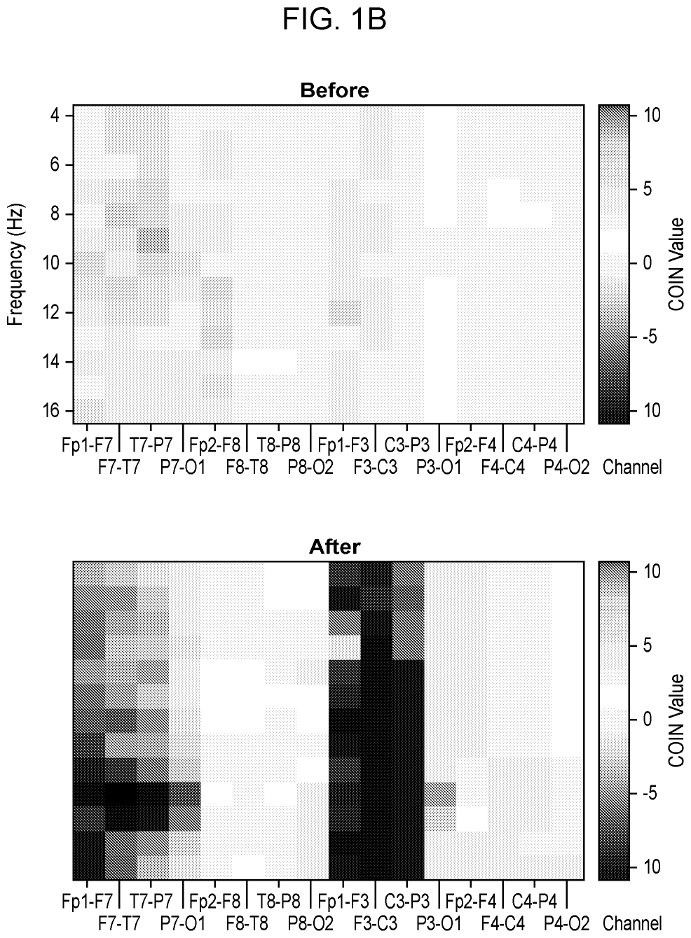

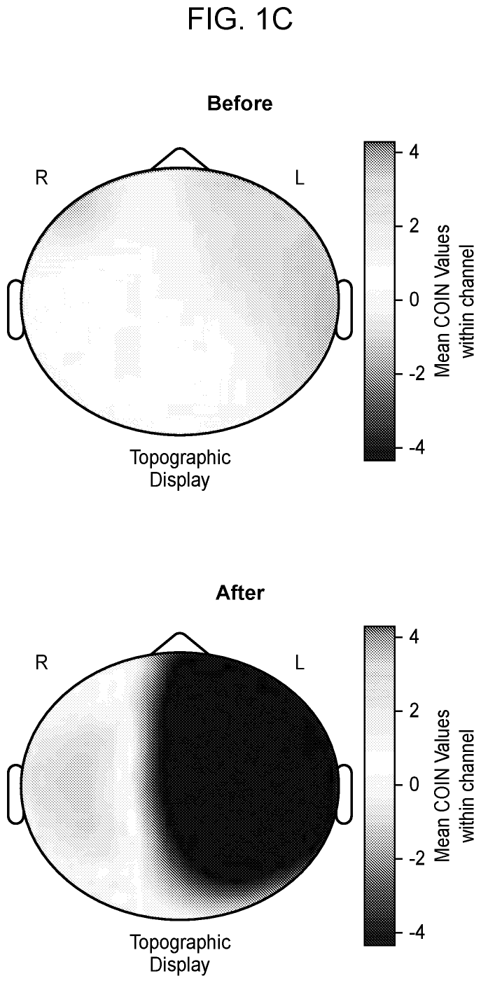

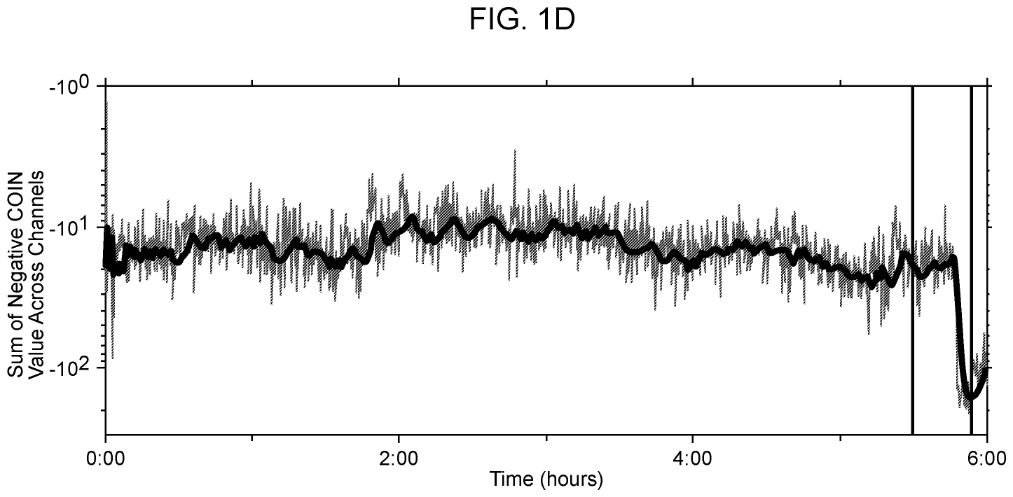

COIN is a quantitative feature derived from raw EEG tracings. FIGS. 1A-1D depicts the EEG preprocessing, artifact identification, and analysis pipeline. Raw EEG files were obtained from the SIPS II study (AIS) or the institution's EEG server. All files were converted to standard European Data Format (.EDF) and deidentified to hash patient name, ID, and offset the study start date. Raw EEG data are time-series data sampled at a minimum 200 Hz and recorded from 19 unipolar channels.

Data were preprocessed on MATLAB using FieldTrip¹⁴ and passed through a 1-70 Hz bandpass filter with a 60 Hz notch filter. The montage was applied and the study was split into 4-second epochs. Artifact detection and rejection was done using Fieldtrip algorithms to detect clipping artifact (amplitude threshold 0.05 uV) and threshold artifact (>300 uV) with z-score rejection for muscle artifact (50-99 Hz, z-threshold 4) and movement artifact (1-5 Hz, z-threshold 4). Epochs containing >2.5 s of artifact were rejected completely, and epochs containing <2.5 s of artifact were rejected partially.¹⁴ Input EEG data are passed through a fast-fourier transform to yield power spectrum data for every 4-second epoch in each channel in a temporal-central-parasagittal (TCP) montage. To analyze data using a limited 10-lead circumferential montage, the EEG processing pipeline was rerun with removal of the parasagittal channels prior to artifact identification and rejection.

### Calculating the Correlate of Injury to the Nervous System (COIN) Index

Raw EEG data are passed through a fast-Fourier transform to yield a power spectrum matrix A where the frequency i=1, 2, . . . , L represents the L-number of power bands inspected and channel j=1, 2, . . . , M represents the M-number of channels inspected. aᵢⱼ thus represents the power for frequency band i on channel j.

Note that frequency i is also referred to herein as frequency f.

Letter “a” with a subscript “j” can refer to an electrode/channel at location “j”. In addition, the letter “a” with subscript “k” is used interchangeably herein with letter “a” with subscript “j*” to refer to a location that is contralateral to a electrode/channel at location “j”.

A matrix R and matrix S are calculated (i & ii) and passed through a cubic function using an elementwise matrix multiplication to generate the COIN matrix Q (iii).

$${r}_{i j} = {log}_{2} ( \frac{{a}_{i j}}{\frac{1}{M} {\sum }_{j = 1}^{M} {a}_{i j}} ) \qquad ( i )$$

$${s}_{i j} = {log}_{2} ( \frac{{a}_{i j}}{{a}_{i k}} ) \qquad ( ii )$$

$$Q = {R}^{2} \cdot S \qquad ( iii )$$

$$where$$

$${q}_{ij} = 0 \qquad ( iv )$$

$${r}_{ij} \cdot {s}_{ij} < 0$$

Q is passed through a gating function to remove instances where rᵢⱼ and sᵢⱼ have opposite polarity (iv).

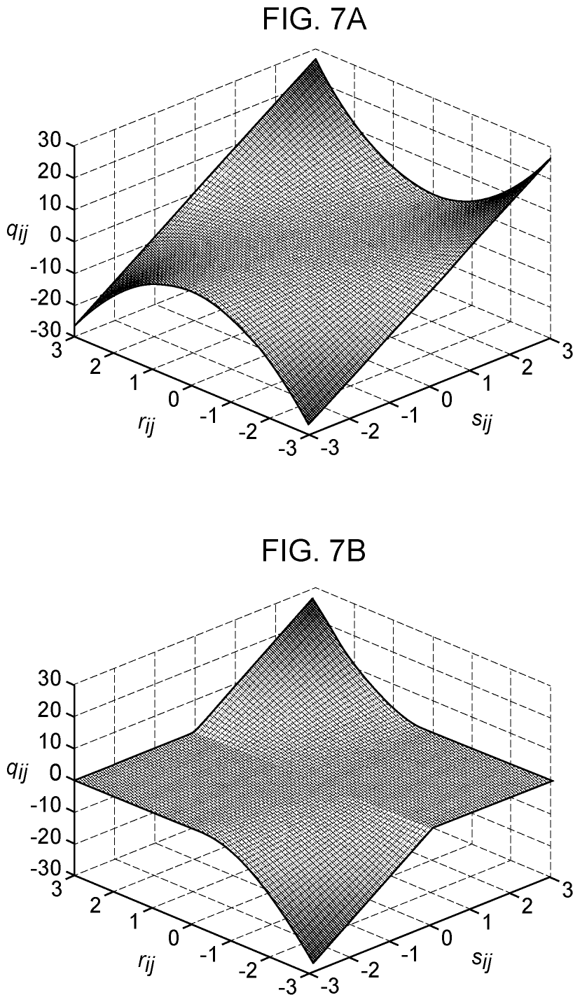

FIG. 7A shows a three-dimensional graph where qᵢⱼ is a function of rᵢⱼ and sᵢⱼ. FIG. 7B shows a three-dimensional graph where qᵢⱼ is a function of r; and sᵢⱼ wherein qᵢⱼ is set to zero when rᵢⱼ multiplied by sᵢⱼ is less than zero, i.e. as shown in equation (iv) above.

FIGS. 1A-1D depicts a sample 4-second epoch of EEG (A) used to generate a COIN-matrix (B). Mean values within each channel are mapped to their corresponding channel locations on a topographic representation of the head to generate the visualizer (C). All channels with negative values are summed to generate a summary value C per 4-second epoch. The values for C are smoothed over time using a 5-minute moving average (D).

$$C = {\sum }_{j = 1}^{M} {\sum }_{i = 4}^{16} {q}_{i j} , for all ( {\sum }_{i = 5}^{16} {q}_{i j} ) < 0$$

### Calculating BSI and ADR

Calculation of BSI was adapted from vanPutten et al., (12) the following equation was applied to the power-spectrum matrix:

$$B S I = \frac{1}{L} {\sum }_{i = 1}^{L} ‖ \frac{1}{M} {\sum }_{j = 1}^{M} \frac{{R}_{i j} − {L}_{i j}}{{R}_{i j} + {L}_{i j}} ‖$$

Where R and L are the channels on the right and left, respectively. The calculation for ADR was adapted using the anterior (F7-T7, F8-T8) and posterior channels (P7-O1, P8-O2) as described by Classen et al.^{8,16} Channels were selected on the temporal chains for the findings to be applicable using a limited circumferential montage. For the analysis, we used the absolute value of the difference between ADR calculated on the left and right.

ADR—the ratio of electrical power between the alpha range (8-13 Hz) and delta range (1-4 Hz)—is a useful tool for cerebral ischemia monitoring as there is relative attenuation of alpha power with an augmentation of delta power under ischemic conditions. The calculation for ADR was adapted using the anterior (F7-T7, F8-T8) and posterior channels (P7-O1, P8-O2) as described by Claassen et al. (Claassen et al., 2004; Rosenthal et al., 2018) The channels were selected on the temporal chains for our findings to be applicable using a portable circumferential EEG device. For the analysis, the absolute value of the difference between ADR calculated on the left and right was used.

### Data Handling and Statistical Analysis

IRB approval was obtained for this project, all EEGs were deidentified prior to analysis. Raw EEG preprocessing and artifact rejection was performed on MATLAB (MathWorks, Natick, MA) using Fieldtrip.¹⁴ Statistical analysis with two-tailed t-test for statistical significance and logistic regression for receiver-operator characteristic (ROC) analysis was performed on MATLAB using the Statistics Toolbox. A Random Forest Classifier (RFC) model was used to accomplish time-window analysis on Python.

The first six hours of EEG data for each subject were used for statistical analysis, the median COIN value over the first six hours of recording were used as individual data points for statistical comparison. Representative neuroimaging (CT or MRI) was compared to topographic visualization of COIN. Two-tailed t-tests were used to compare continuous variables and logistic regression to derive area under receiver-operator characteristic (AUROC) curves. The Youden J Statistic (sensitivity+specificity−1) was calculated to determine ideal cutoff point for optimizing test characteristics. Sensitivity, specificity, and test accuracy were calculated by applying the optimal cutoff points to median COIN values from each recording. Analysis was performed on MATLAB 2021b using the Statistics Toolbox.

One of the secondary goals of this study was to determine how much EEG data was needed for stroke prediction using COIN. First, 5-minute long clips were created that were randomly sampled from each subjects' EEG recording. A vector containing the proportion of time spent below COIN values for a range of COIN values between −5 and −35 for this 5-minute epoch. Vectors from each stroke and control subject were compiled to generate a matrix which was used as input data for a Random Forest Classifier (RFC) model to predict stroke diagnosis (Python version 3.9.1 and scikit-learn). (Pedregosa et al., 2011) This process was repeated 200 times using different randomly selected time-clips from each patient for each iteration. Aggregate performance data (sensitivity, specificity, accuracy and area under the receiver operating curve [AUROC]) were compiled. This process was systematically repeated using clips of increasing length starting at 5-minute duration and up to 30 minutes (i.e., the first 200 RFC models were of EEG clips with 5-minute duration each, the following 200 models with 6-minute length, and so-on until reaching 30 minute clips). This procedure was performed to evaluate how model performance changed as longer clips of EEG data were used. Mean and standard deviation of accuracy, AUROC, sensitivity, and specificity were reported for each time window.

### Results

90 patients in the SIPS cohort were screened and 23 patients met inclusion criteria. One patient was excluded for presence of hemorrhage, and 2 patients were excluded for poor EEG quality. Twenty stroke patients (5.2±5.5 years of age, 20% female) and 29 control patients age (4.9±5.8 years of age, 52% female) were analyzed.

Radiographic stroke localization was anterior circulation (AC) stroke in 60% (n=12) and posterior circulation (PC) stroke in 40% (n=8). RIV was >5% in 35% (n=7) and >10% in 60% (n=12). Stroke was on the left in 45% (n=9), on the right in 30% (n=6), and bilateral in 25% (n=5). Mean RIV was 8.1%, with a range of 0.01%-22%. Imaging was obtained before EEG in 55% of patients (n=11)

Mean time to EEG placement was 17±13 hours. Fifty-five percent of patients (n=11) had a seizure within 14 days of stroke and 35% (n=7) had seizure captured on EEG. EEG was read as normal in 15% (n=3) of patients, abnormal with epileptiform findings in 35% (n=7), and abnormal with non-epileptiform findings in 50% (n=10). Antiseizure medications (ASMs) were given to 55% (n=11) of patients with 10% (n=2) receiving one, 20% (n=4) receiving two, and 25% (n=5) receiving three or more ASMs. Forty-five percent (n=9) of patients were not given an ASM. Imaging was obtained before EEG in 55% (n=11) of patients. Patient characteristics are summarized in Table 1, which is shown below.

| | Stroke | | Control | | Control | |
|---|---|---|---|---|---|---|
| Variable | (n = 20) | | (n = 29) | | (n = 14) | |
| Age, y (SD) | 5.2 | (5.5) | 4.9 | (5.8) | 9.8 | (6.6) |
| Sex, n Female (%) | 4 | (20) | 15 | (52) | 8 | (57) |
| Arterial Territory of Infarct, n (%) | | | | | | |
| Anterior Circulation | 12 | (60) | — | — | — | — |
| Posterior Circulation | 8 | (40) | — | — | — | — |
| Watershed | 3 | (15) | — | — | — | — |
| Laterality of Infarct, n (%) | | | | | | |
| Left | 9 | (45) | — | — | — | — |
| Right | 6 | (30) | — | — | — | — |
| Bilateral | 5 | (25) | — | — | — | — |
| Relative Infarct Volume, % (range) | 8.1 | (0.01-22) | — | — | — | — |
| Mean time to EEG after Stroke, h (SD) | 17 | (13) | — | — | — | — |
| Seizure within 14 d of stroke, n (%) | 11 | (55) | — | — | — | — |
| EEG Result, n (%) | | | | | | |
| Normal | 3 | (15) | 29 | (100) | 4 | (29) |
| Abnormal, Epileptiform | 7 | (35) | 0 | (0) | 0 | (0) |
| Abnormal, Nonepileptiform | 10 | (50) | 0 | (0) | 10 | (71) |
| Electrographic seizures, n (%) | 7 | (35) | 0 | (0) | 0 | (0) |
| Number of Sedative or Antiseizure | | | | | | |
| Medications, n (%) | | | | | | |
| None | 9 | (45) | 0 | (0) | 7 | (50) |
| One | 2 | (10) | 0 | (0) | 3 | (21) |
| Two | 4 | (20) | 0 | (0) | 2 | (14) |
| Three or More | 5 | (25) | 0 | (0) | 2 | (14) |

### Topographic Visualization of COIN

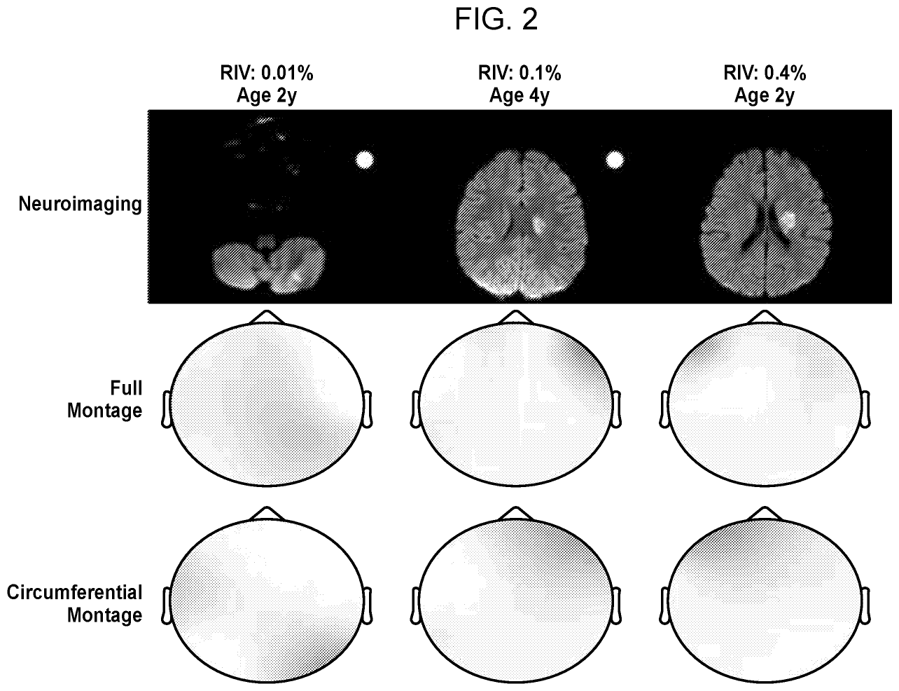

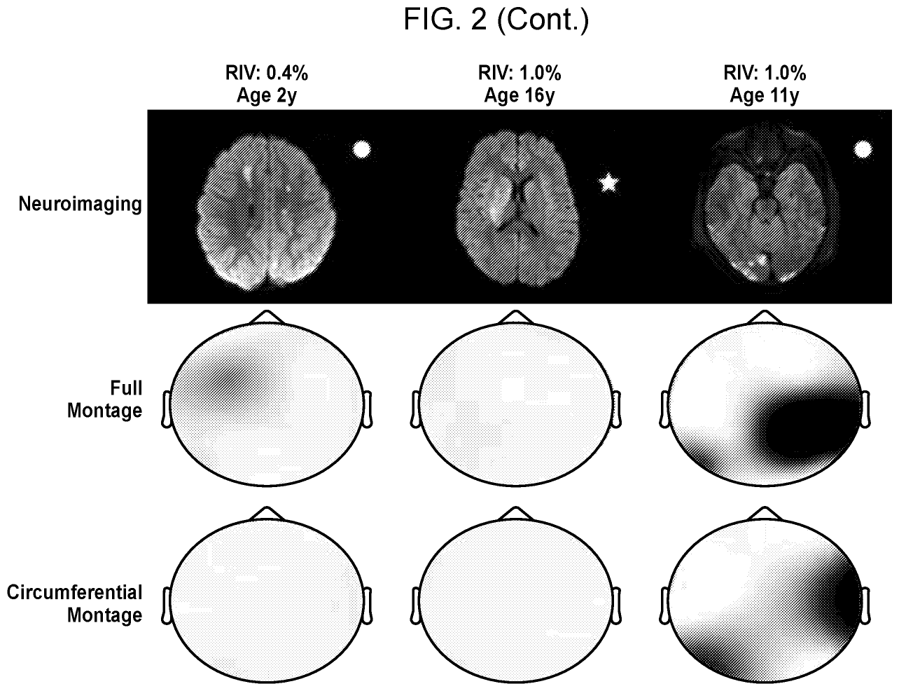

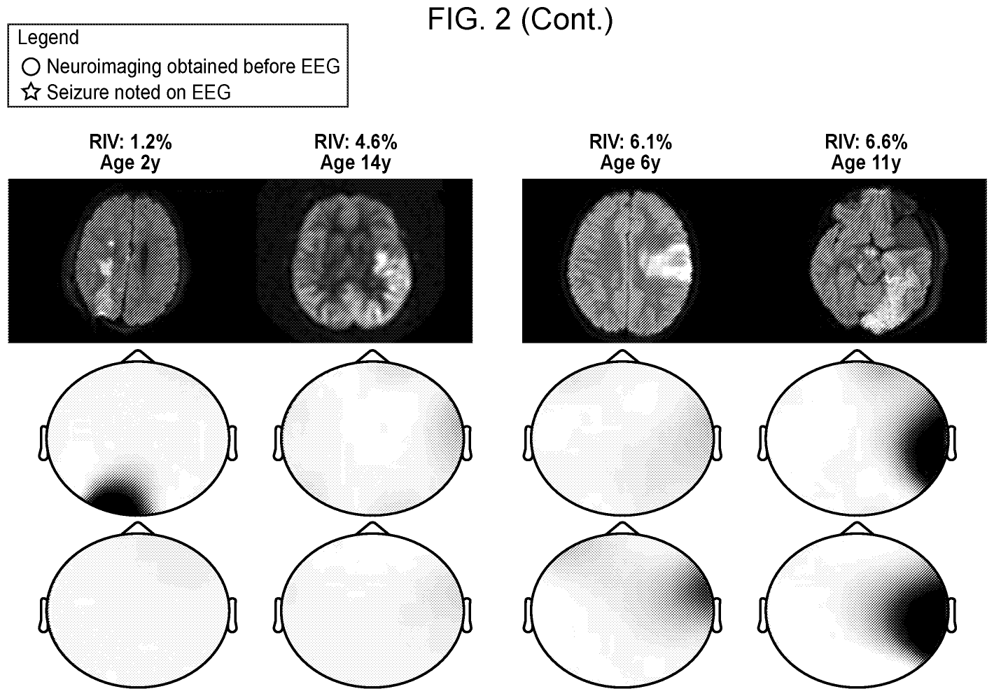

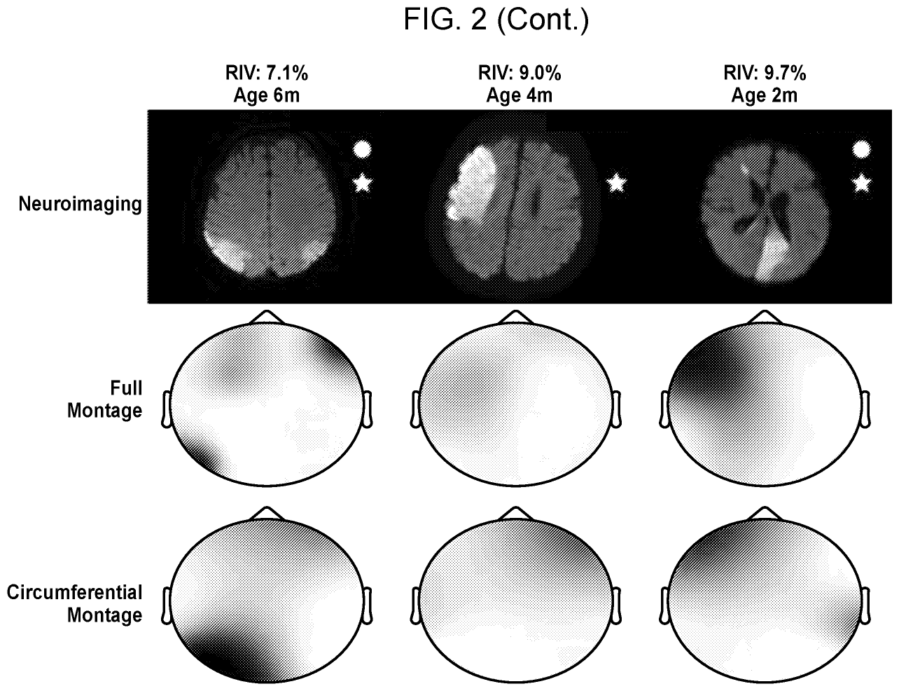

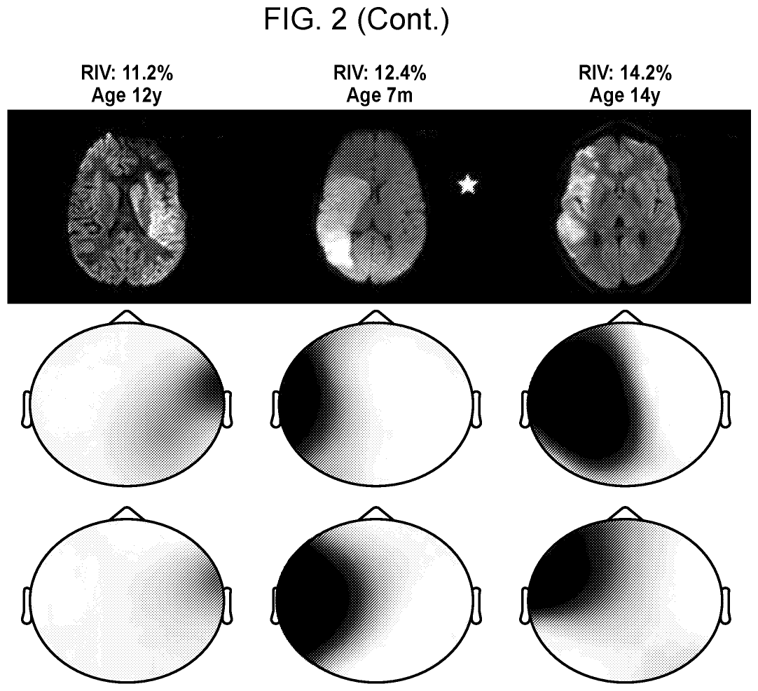

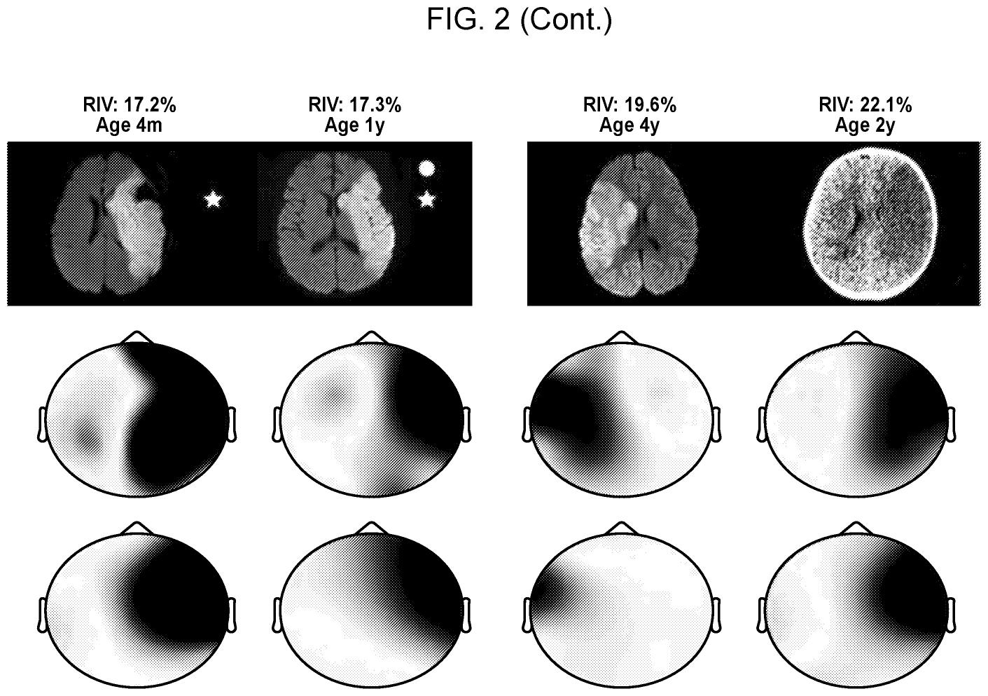

FIG. 2 shows comparison of representative neuroimages for each stroke patient to topographic representations of COIN using a full montage as well as a limited circumferential montage. Patients with seizure captured on EEG and those who had imaging before EEG are indicated.

### Test Characteristics of the Summary COIN Value, BSI, and ADR

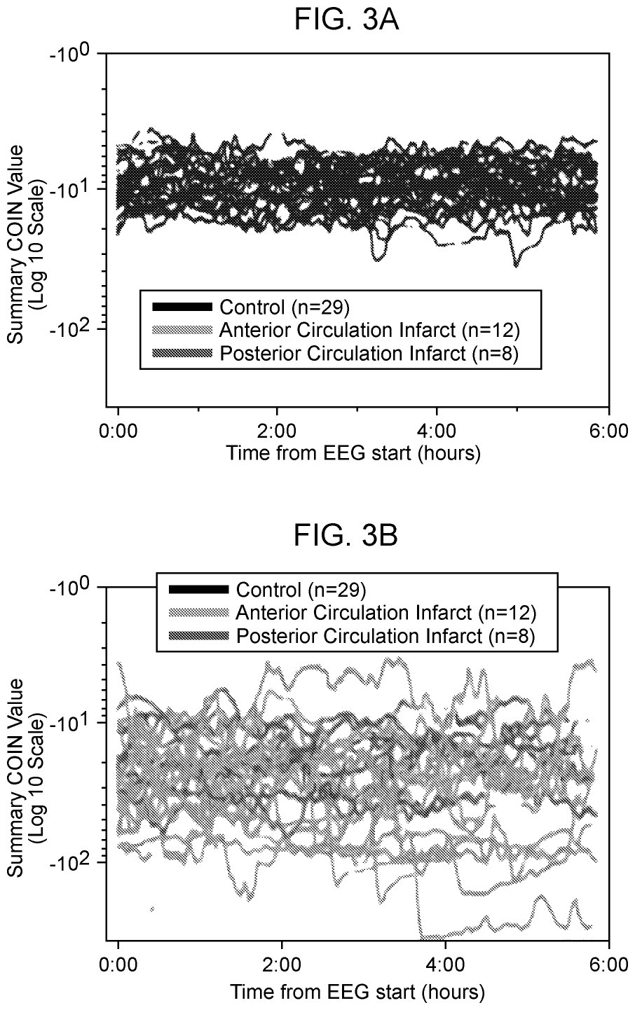

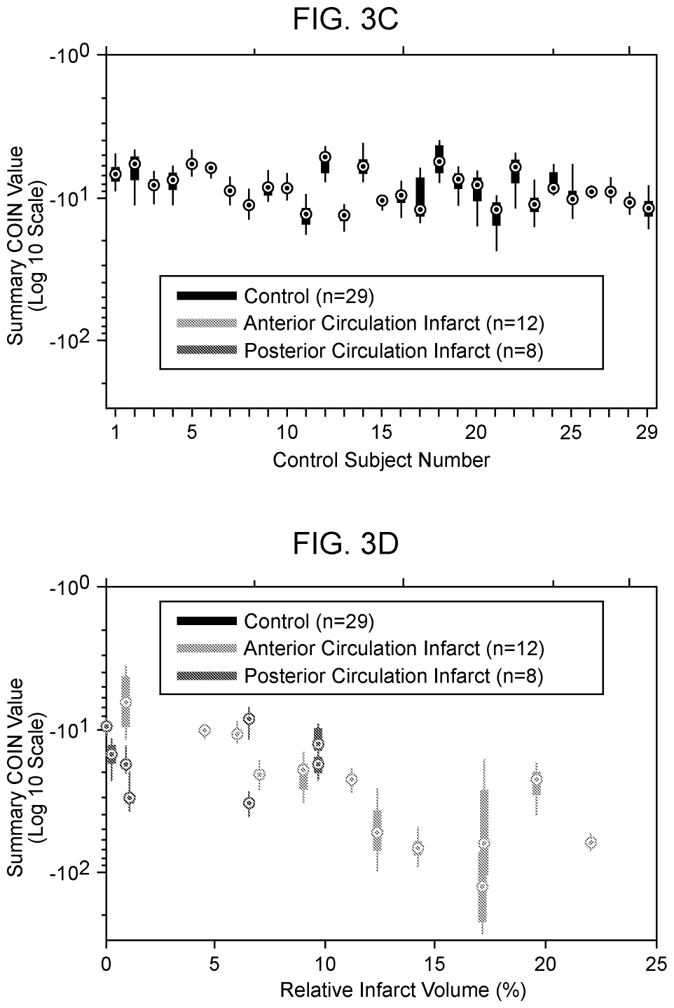

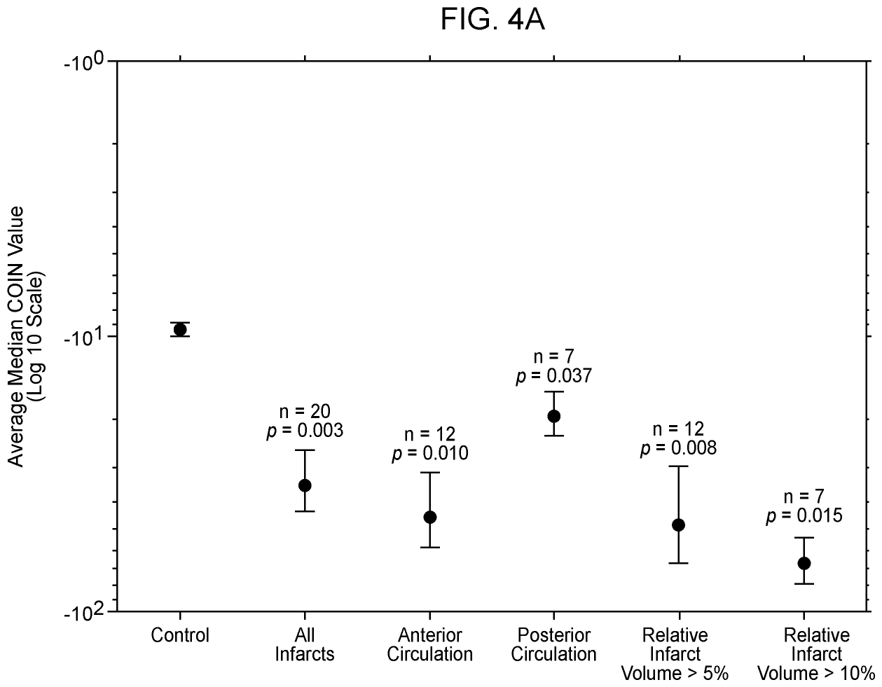

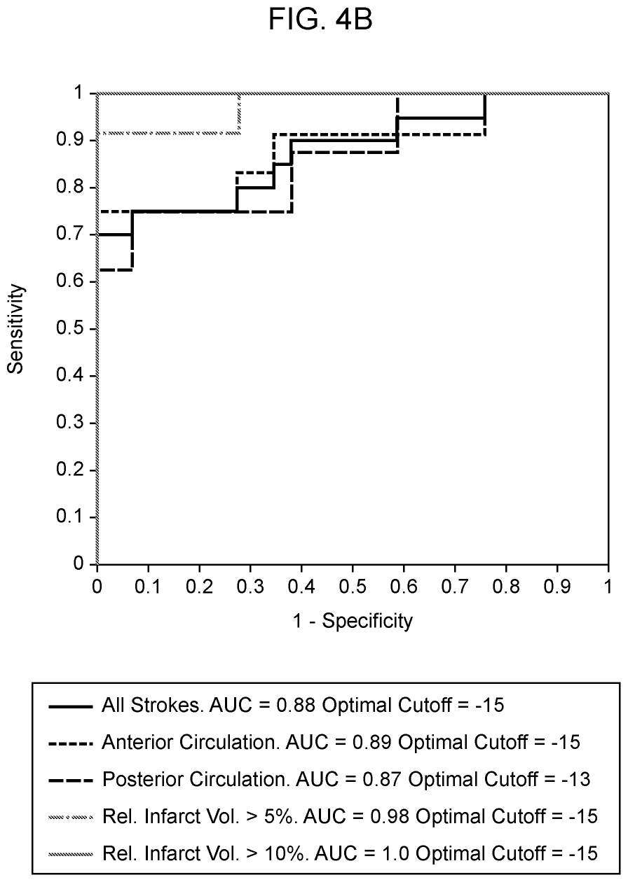

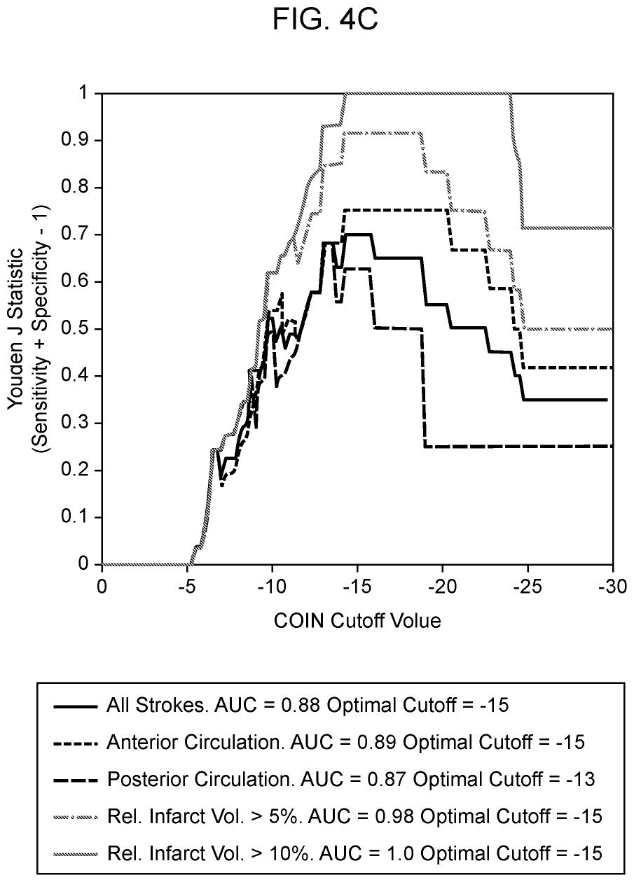

Median values from the first 6 hours of EEG recording from stroke and control patients were included for statistical analysis (FIGS. 3A-3D). Average median COIN was −34.7 in the primary cohort of all stroke patients and −9.5 in controls (p=0.013) with an area under ROC of 0.88, sensitivity and specificity of 70% and 100%, and an optimal COIN cutoff value of −15. FIGS. 4A-4C shows performance statistics in the primary cohort, as well as subgroups composed of patients with anterior circulation strokes, posterior circulation strokes, strokes with RIV >5% and strokes with RIV >10%. Table 2 summarizes results using COIN in full and circumferential montage, BSI, and ADR. Table 2 is shown below in two parts, i.e. Table 2A and Table 2B.

TABLE 2A

| | Mean of | SE of | | |
|---|---|---|---|---|
| | Median | Median | p- | Optimal |
| | Values | Values | Value | Cutoff |
| COIN Full Montage | | | | |
| Control | −9.5 | −0.5 | | |
| All Strokes | −34.6 | −8.7 | 0.003 | −15 |
| Anterior Circulation | −44.9 | −13.5 | 0.011 | −15 |
| Posterior Circulation | −19.4 | −3.6 | 0.026 | −13 |
| Relative Infarct Vol. >5% | −48 | −18.5 | 0.005 | −15 |
| Relative Infarct Vol. >10% | −66.6 | −12.9 | 0.009 | −15 |
| COIN Circumferential Montage | | | | |
| Control | −3.9 | −0.3 | | |
| All Strokes | −16.9 | −4.6 | 0.009 | −7.5 |
| Anterior Circulation | −22.3 | −7.1 | 0.02 | −6 |
| Posterior Circulation | −8.9 | −3.3 | 0.18 | −7.5 |
| Relative Infarct Vol. >5% | −25.1 | −10.4 | 0.007 | −7.5 |
| Relative Infarct Vol. >10% | −33.4 | −6.8 | 0.02 | −7.5 |
| Brain Symmetry Index | | | | |
| Control | 0.106 | −0.005 | | |
| All Strokes | 0.204 | −0.023 | <0.001 | 0.107 |
| Anterior Circulation | 0.24 | −0.033 | 0.001 | 0.145 |
| Posterior Circulation | 0.15 | −0.016 | 0.035 | 0.135 |
| Relative Infarct Vol. >5% | 0.257 | −0.033 | <0.001 | 0.145 |
| Relative Infarct Vol. >10% | 0.311 | −0.028 | <0.001 | 0.17 |
| Anterior ADR Difference | | | | |
| Control | 0.012 | −0.002 | | |
| All Strokes | 0.026 | −0.005 | 0.01 | 0.014 |
| Anterior Circulation | 0.033 | −0.008 | 0.03 | 0.027 |
| Posterior Circulation | 0.017 | −0.005 | 0.15 | 0.014 |
| Relative Infarct Vol. >5% | 0.027 | −0.009 | 0.07 | 0.014 |
| Relative Infarct Vol. >10% | 0.027 | −0.007 | 0.15 | 0.014 |
| Posterior ADR Difference | | | | |
| Control | 0.019 | −0.006 | | |
| All Strokes | 0.023 | −0.007 | 0.8 | 0.009 |
| Anterior Circulation | 0.03 | −0.011 | 0.51 | 0.009 |
| Posterior Circulation | 0.012 | −0.003 | 0.2 | 0.008 |
| Relative Infarct Vol. >5% | 0.023 | −0.002 | 0.75 | 0.009 |
| Relative Infarct Vol. >10% | 0.012 | −0.009 | 0.19 | 0.009 |

TABLE 2B

| | | | | AUROC | AUROC |
|---|---|---|---|---|---|
| | Sensitivity | Specificity | Accuracy | (95% CI) | (95% CI) |
| COIN Full Montage | | | | | |
| Control | | | | | |
| All Strokes | 70% | 100% | 88% | 0.88 | (0.74-0.96) |
| Anterior Circulation | 75% | 100% | 93% | 0.89 | (0.66-0.98) |
| Posterior Circulation | 75% | 93% | 89% | 0.87 | (0.59-0.99) |
| Relative Infarct Vol. >5% | 92% | 100% | 98% | 0.98 | (0.87-1) |
| Relative Infarct Vol. >10% | 100% | 100% | 100% | 1 | (1-1) |
| COIN Circumferential Montage | | | | | |
| Control | | | | | |
| All Strokes | 60% | 100% | 84% | 0.83 | (0.65-0.93) |
| Anterior Circulation | 92% | 90% | 90% | 0.94 | (0.78-0.99) |
| Posterior Circulation | 38% | 100% | 86% | 0.66 | (0.37-0.90) |
| Relative Infarct Vol. >5% | 92% | 100% | 98% | 0.99 | (0.92-1) |
| Relative Infarct Vol. >10% | 100% | 100% | 100% | 1 | (1-1) |
| Brain Symmetry Index | | | | | |
| Control | | | | | |
| All Strokes | 95% | 69% | 80% | 0.89 | (0.75-0.95) |
| Anterior Circulation | 83% | 90% | 88% | 0.94 | (0.81-0.99) |
| Posterior Circulation | 75% | 83% | 81% | 0.81 | (0.58-0.93) |
| Relative Infarct Vol. >5% | 100% | 90% | 93% | 0.99 | (0.93-1) |
| Relative Infarct Vol. >10% | 100% | 100% | 100% | 1 | (1-1) |
| Anterior ADR Difference | | | | | |
| Control | | | | | |
| All Strokes | 70% | 72% | 71% | 0.7 | (0.51-0.84) |
| Anterior Circulation | 42% | 100% | 83% | 0.71 | (0.48-0.89) |
| Posterior Circulation | 75% | 72% | 73% | 0.69 | (0.34-0.87) |
| Relative Infarct Vol. >5% | 67% | 72% | 71% | 0.72 | (0.39-092) |
| Relative Infarct Vol. >10% | 71% | 72% | 72% | 0.69 | (0.43-0.86) |
| Posterior ADR Difference | | | | | |
| Control | | | | | |
| All Strokes | 70% | 55% | 61% | 0.54 | (0.36-0.70) |
| Anterior Circulation | 75% | 55% | 61% | 0.57 | (0.36-0.77) |
| Posterior Circulation | 75% | 48% | 54% | 0.5 | (0.30-0.72) |
| Relative Infarct Vol. >5% | 75% | 55% | 61% | 0.55 | (0.29-0.76) |
| Relative Infarct Vol. >10% | 71% | 55% | 58% | 0.49 | (0.35-0.74) |

As shown in the tables above, stroke was detected with a sensitivity of 70%, a specificity of 100%, and an accuracy of 88% (see: Table 2B3: COIN Full Montage: All Strokes). Additionally, stroke with relative infarct volume of >5% was detected with sensitivity of 92%, specificity of 100%, and accuracy of 98% (Table 2B: COIN Full Montage: Relative Infarct Volume >5%). The threshold values for C used to achieve these detections was −15 in both cases (see Table 2A: Optimal Cutoff column).

### Time Window Analysis

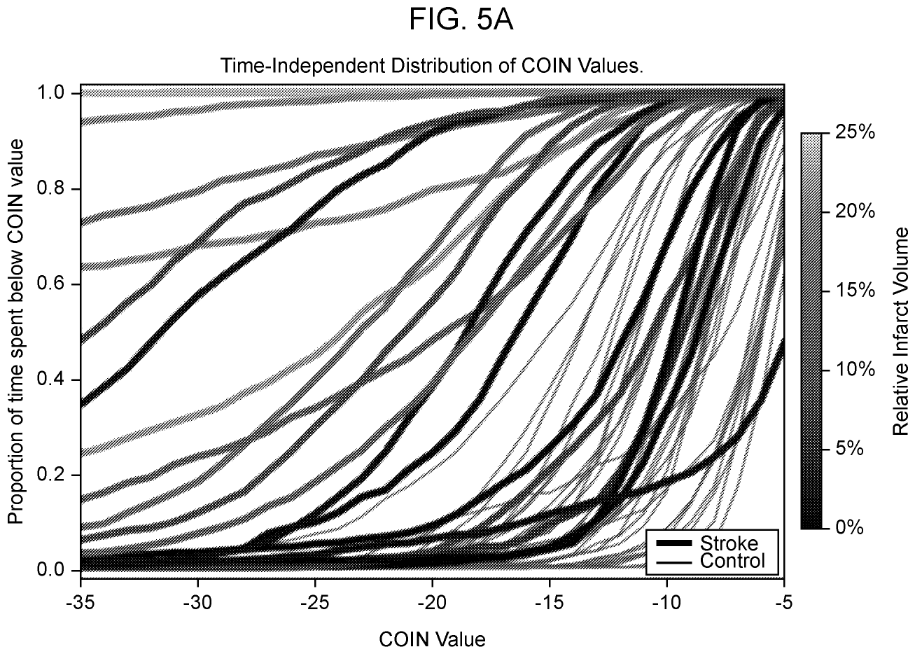

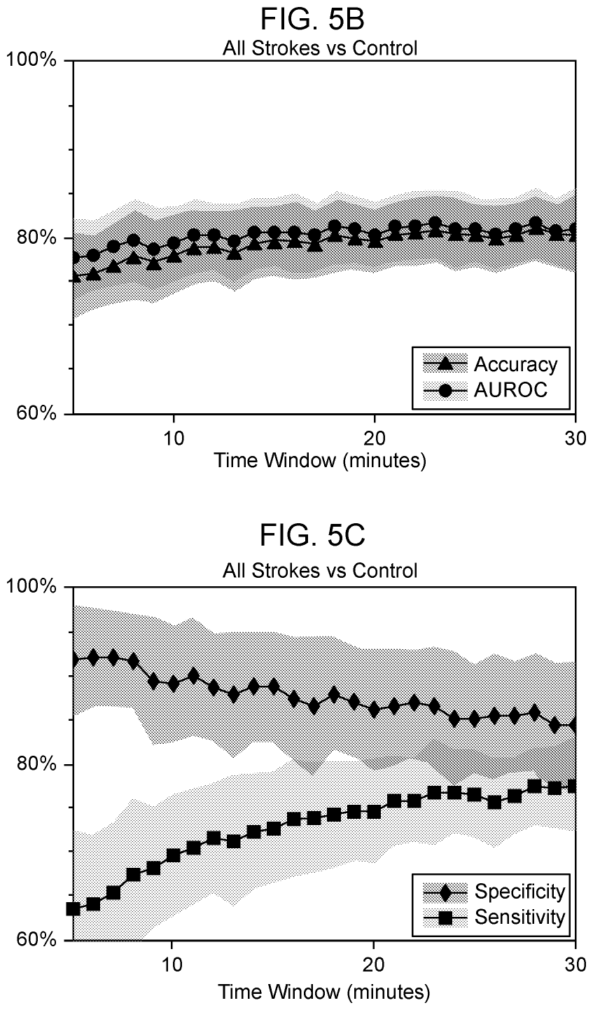

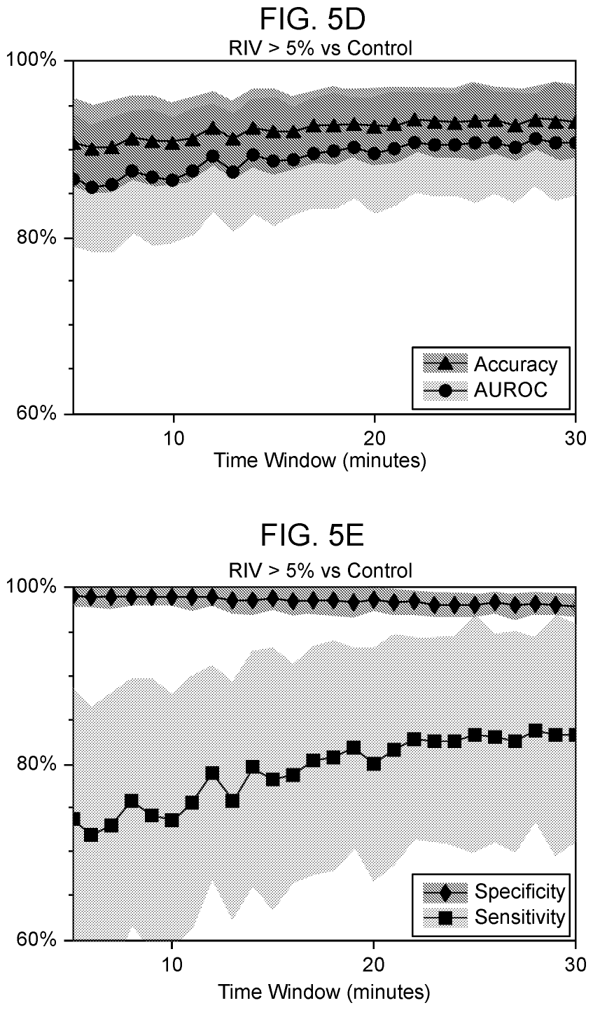

COIN data for stroke and control were expressed as a time-independent proportion of the study spent below COIN cutoffs ranging from −35 to −5 (FIG. 5A), which were used as input data for a Random Forest Classification model. Two-hundred random time windows ranging from 5 to 30 minutes in length were used to measure accuracy, AUROC, sensitivity, and specificity. Results of the time-window analysis are shown in FIGS. 5B-5E.

### Discussion

This study demonstrates that a continuous and quantitative measure of brain asymmetry derived from scalp EEG data has high sensitivity and specificity for detecting acute strokes with >5% relative infarct volume in hospitalized children. There has been limited validation of QEEG algorithms for stroke detection in children, and this study has shown that the discriminative performance of COIN was superior to ADR and comparable to BSI, with the added benefit of providing an interpretable visualization to localize the region of asymmetry in the brain. Moreover, the performance of COIN in detecting stroke using full and limited (10 electrodes) EEG montage was comparable for anterior circulation stroke, suggesting that this diagnostic technology has the potential to be deployed using portable EEG monitors that do not require expert technologist placement.

The topographic visualizer showed concordance with imaging in localizing the area of infarct for most patients, however for some patients there was discordance in the magnitude of the COIN signal and the infarct volume on imaging. For subjects with small infarcts on imaging and large COIN signals, the imaging tended to occur before EEG suggesting that the infarct territory might have expanded since the imaging. Conversely, for patients with large infarcts on imaging but small COIN signals, the imaging tended to occur after the EEG was removed. This may also be explained by an expansion of the infarct territory occurred from the first six hours of recording to the point of imaging. This result highlights the dynamic nature of COIN and will require future evaluation.

It was determined that a COIN cutoff value of −15 could be used to discriminate stroke from non-stroke using the Youden J statistic. This finding was consistent across subgroups of stroke volume and vascular territory and the COIN value was more prominent with larger stroke volumes. BSI showed comparable performance in discriminating stroke from controls to COIN, however it had higher variability in optimal cutoff values across stroke volume and vascular territory subgroups. Moreover, BSI does not provide localization or lateralization data, and thus cannot be used to generate a topographical display.^{12,17}

The quantitative stroke metric ADR relies on a loss of alpha power in conjunction with an increase of delta power, where a resultant decrease in the ADR implies ischemia of the underlying brain tissue. It was found that ADR performed poorly in children, possibly in part due to a difference in the EEG background frequency admixture in infants compared to older children and adults. The relative underrepresentation of activity above 8 Hz in children younger than 1 year precludes the use of ADR which relies on the presence of electrical activity in the alpha (8-12 Hz) range.¹⁸ ADR also showed a worsening ability to discriminate large strokes from control patients. This is likely explained by the loss of delta power that occurs after a complete loss of regional cortical perfusion leading to an increase (or pseudo-normalization) of the ADR. ADR has been shown to be useful for dynamic detection and trending of cerebral ischemia in subarachnoid hemorrhage,^{8,16} yet the results suggest it may not be a useful screening tool for detecting large strokes.

The results of the time window analysis demonstrate improvement in COIN accuracy and sensitivity as the recording period increases from 5 minutes until 20 minutes, with a subsequent plateau in performance. This finding suggests that changes in COIN could be detected rapidly after the onset of stroke and in time for confirmatory testing and therapeutic interventions. While improvements were seen in accuracy and sensitivity with increasing time-windows, it was observed a slight reduction in test specificity over time. It is hypothesized that this to be reflective of the random forest classification model being designed to optimize accuracy, resulting in a larger COIN cutoff with shorter time windows that minimizes the false positive rate. For strokes >5% RIV, the model was able to maintain a specificity >95% with a 5-minute recording suggestive that a very large COIN value could be used to diagnose large strokes very rapidly with a low false-positive rate.

The accuracy of COIN using a circumferential montage demonstrates its potential as a rapid triage tool using a limited EEG setup. Use of limited circumferential EEG has been shown to reduce time to diagnosis of subclinical seizures and was associated with cost-savings and reduction in patient length of stay in the acute care and intensive care setting.^{19,20} A similar approach could be developed for rapid triage and identification of stroke in the pre-hospital setting, or in certain clinical scenarios such as cardiac catheterization²¹ and mechanical circulatory support.^{6,22}

A point-of-care brain monitoring algorithm for stroke detection could streamline workflow for acute stroke management in hospitalized children, particularly those at high risk for stroke after cardiac procedures or during extracorporeal life support when there is often a limited or unreliable neurological exam due to sedation or muscular blockade. Importantly, COIN's high sensitivity and specificity for large strokes would also support decisions about risk and benefit of transporting critically ill patients to the CT scanner and exposing them to radiation, a common dilemma faced by intensivists and nursing teams managing patients at risk for stroke and other catastrophic neurological emergencies.

The rationale for EEG as a sensitive marker for brain ischemia is clearly described,²³ however there has been limited investigation on deploying continuous EEG monitoring for stroke detection in children despite a large body of work in the adult population.⁹ While there are no prospective trials in EEG detection of stroke, a small pilot study in adult patients demonstrated feasibility of EEG for detection of ischemic stroke in the emergency department.²⁴ EEG is considered an ideal platform for detection of stroke with high diagnostic accuracy¹³ and is generally suggested in patients with known neurological injuries who are at high risk of worsening ischemia,²⁵ however it is limited by the requirement of expert interpretation. Implementation of COIN in various clinical contexts may help overcome this feasibility barrier by making the detection of ischemia on EEG accessible to non-epileptologists.

This pilot study was possible in the pediatric population due to the high prevalence of seizures in pediatric stroke, and the frequent use of EEG as a result.¹⁴ While the prevalence seizures and ASMs in the stroke cohort may have affected the EEG background, seizures were likely to have contributed to a small portion of the background recording as they were brief and treated. The performance of the COIN algorithm for stroke detection was not significantly affected by the presence of seizures or use of ASMs. The SIPS EEG dataset was not designed for real-time capture of stroke, as EEG was placed after stroke onset in all cases. Furthermore, the timing of stroke was dependent on the time the patient was last seen normal or positive diagnostic imaging was obtained, neither of which are precise reflections of the time of stroke onset.

The results do not account for the patients' neurological exam, and thus it was not possible to assess the utility of COIN compared to a clinical diagnosis of stroke, or how COIN relates to the severity of any abnormal neurological exam. The use of EEG to confirm a diagnosis of stroke in a patient who presents with an obvious stroke syndrome is likely to be of limited value. Future study of COIN will need to assess how it performs within specific clinical scenarios where it would be expected to enhance clinical diagnosis such as in the periprocedural window or for patients who are sedated or under neuromuscular blockade in the intensive care unit.

### REFERENCES

12. Vanputten M. Extended BSI for continuous EEG monitoring in carotid endarterectomy. Clinical Neurophysiology. 2006; 117(12):2661-2666. doi:10.1016/j.clinph.2006.08.007
13. van Meenen L C C, van Stigt M N, Siegers A, et al. Detection of Large Vessel Occlusion Stroke in the Prehospital Setting: Electroencephalography as a Potential Triage Instrument. Stroke. 2021; 52(7). doi:10.1161/STROKEAHA.120.033053
14. Fox C K, Mackay M T, Dowling M M, et al. Prolonged or recurrent acute seizures after pediatric arterial ischemic stroke are associated with increasing epilepsy risk. Dev Med Child Neurol. 2017; 59(1):38-44. doi:10.1111/dmcn.13198
15. Oostenveld R, Fries P, Maris E, Schoffelen J M. FieldTrip: Open Source Software for Advanced Analysis of MEG, EEG, and Invasive Electrophysiological Data. Computational Intelligence and Neuroscience. 2011; 2011:1-9. doi:10.1155/2011/156869
16. Claassen J, Hirsch L J, Kreiter K T, et al. Quantitative continuous EEG for detecting delayed cerebral ischemia in patients with poor-grade subarachnoid hemorrhage. Clinical Neurophysiology. 2004; 115(12):2699-2710. doi:10.1016/j.clinph.2004.06.017
17. Wilkinson C M, Burrell J I, Kuziek J W P, Thirunavukkarasu S, Buck B H, Mathewson K E. Predicting stroke severity with a 3-min recording from the Muse portable EEG system for rapid diagnosis of stroke. Sci Rep. 2020; 10(1):18465. doi:10.1038/s41598-O20-75379-w
18. A Kaminska, M Eisermann, P Plouin. Child EEG (and maturation). Handbook of Clinical Neurology. 2019; 160:125-142. doi:10.1016/B978-0-444-64032-1.00008-4
19. Ney J P, Gururangan K, Parvizi J. Modeling the economic value of Ceribell Rapid Response EEG in the inpatient hospital setting. Journal of Medical Economics. 2021; 24(1):318-327. doi:10.1080/13696998.2021.1887877
20. LaMonte M P. Ceribell EEG shortens seizure diagnosis and workforce time and is useful for COVID isolation. Epilepsia Open. 2021; 6(2):331-338. doi:10.1002/epi4.12474
21. Harrar D B, Salussolia C L, Vittner P, et al. Stroke After Cardiac Catheterization in Children. Pediatric Neurology. 2019; 100:42-48. doi:10.1016/j.pediatrneurol.2019.07.005
22. Di Gennaro J L, Chan T, Farris R W D, Weiss N S, McMullan D M. Increased Stroke Risk in Children and Young Adults on Extracorporeal Life Support with Carotid Cannulation. ASAIO Journal. 2019; 65(7):718-724. doi:10.1097/MAT.0000000000000912
23. Jordan K G. Emergency EEG and Continuous EEG Monitoring in Acute Ischemic Stroke. Journal of Clinical Neurophysiology. 2004; 21(5):12.
24. Shreve L, Kaur A, Vo C, et al. Electroencephalography Measures are Useful for Identifying Large Acute Ischemic Stroke in the Emergency Department. Journal of Stroke and Cerebrovascular Diseases. 2019; 28(8):2280-2286. doi:10.1016/j.jstrokecerebrovasdis.2019.05.019

### Example 2: Differentiating Large Versus Small Strokes in Adults

### Overview of Methods

Retrospective cohort assessing performance of COIN in adult patients at a single university-affiliated hospital with ischemic stroke using a convenience dataset containing 8 hours of EEG data per subject. Subjects are categorized as having large or small stroke based on a threshold volume of 100 mL. COIN is calculated in separate 4-second epochs by cross referencing power ratios in each channel relative to the entire field and to the contralateral homologue. COIN data are used to visualize stroke territory, and random forest classification with 10-fold cross-validation task is used to obtain test performance metrics. To assess length of required EEG to optimize performance, analysis is repeated using pooled restricted samples from random time windows ranging 5 to 30 minutes.

### Overview of Results

Thirty-five patients with mean age 67 (SD 17) years and median NIHSS 14 (IQR 8-19) were analyzed. Ten patients had large stroke and 25 had a small stroke. Average (SE) of median COIN values were −53 (13) in the large and −16 (1.2) in the small group (p=0.0001). Average test performance using 8 hours of recording per patient was accuracy 86%, sensitivity 90%, specificity 84%.

### Overview of Conclusions

COIN can differentiate large (core volume >100 mL) from small ischemic strokes with good accuracy and high specificity. Prospective implementation and evaluation are required to determine utility of COIN as an aid to stroke diagnosis in patients with a limited exam.

### Study Population

Retrospective secondary analysis of EEG and clinical data from patients >18 years with AIS and at least 8 h of continuous EEG at a single center from 2012 to 2019. Patients were excluded for absence of neuroimaging and for EEG recordings with excessive line interference, muscle artifact, burst suppression, or severe voltage suppression. Neuroimaging was available as representative images previously stored for each patient. Clinical data included age and sex of patients, stroke laterality and location, NIH Stroke Scale (NIHSS), TOAST score, and stroke volume calculated by ABC/2.

### EEG Processing

A bipolar montage was applied, and the study was split into 4-second epochs. Artifact detection and rejection was performed using algorithms in Fieldtrip to detect clipping artifact (amplitude threshold 0.05 uV) and threshold artifact (>300 uV) with z-score rejection for muscle artifact (50-99 Hz, z-threshold=4) and movement artifact (1-5 Hz, z-threshold=4). Each epoch containing >2.5 s of artifact was rejected, and epochs containing <2.5 s of artifact were rejected partially to allow remaining usable data from the epoch to be analyzed. 15 Input EEG data were passed through a fast-fourier transform to yield power spectrum data for every 4-second epoch in each channel in the bipolar montage.

### Calculating the Correlate of Injury to the Nervous System (COIN) Index

Raw EEG data are passed through a fast-Fourier transform to yield a power spectrum matrix A which is input into equations (i) through (iv) below:

$${r}_{i j} = {log}_{2} ( \frac{{a}_{i j}}{\frac{1}{M} {\sum }_{j = 1}^{M} {a}_{i j}} ) \qquad ( i )$$

$${s}_{i j} = {log}_{2} ( \frac{{a}_{i j}}{{a}_{i k}} ) \qquad ( ii )$$

$$Q = {R}^{2} \cdot S \qquad ( iii )$$

$${q}_{ij} = 0 \qquad ( iv )$$

$$Where$$

$${r}_{ij} \cdot {s}_{ij} < 0$$

Mean values within each channel are mapped to a topographic visualizer, and all channels with negative values are summed to generate a summary COIN value C per 4-second epoch. The values for C are smoothed over time using a 5-minute moving average. For the present study, we chose a frequency range of 8-18 Hz to mirror the attenuative effects of stroke predominantly in the alpha power band (8-13 Hz) in adults.

$$C = {\sum }_{j = 1}^{M} {\sum }_{i = 8}^{18} {q}_{i j} , for all ( {\sum }_{i = 4}^{16} {q}_{i j} ) < 0$$

### Data Handling and Statistical Analysis

Raw EEG preprocessing and artifact rejection was performed on MATLAB (MathWorks, Natick, MA) using Fieldtrip. 15 Eight hours of EEG recording were available from each patient for analysis. Median COIN values over the entire recording were used as individual data points for two-sample t-test and logistic regression.

### Results

Patient Characteristics and Neuroimaging findings. Of 71 patients, 35 met inclusion and exclusion criteria with mean (SD) age 67(17) and NIHSS 13.7(6.6). Infarct volume was <100 mL in 25 patients (age 69+/−18 years, 48% female, NIHSS 12.9+/−6.8, mean stroke volume 44+/−24) and >100 mL in 10 patients (age 62+/−15 years, 50% female, NIHSS 16.1+/−5.5, mean stroke volume 233+/−108). For patients with infarct volume <100 mL and >100 mL, stroke laterality was left in 16 (64%) and 5 (50%), right in 4 (16%) and 4 (40%), bilateral in 2 (8%) and 1(10%), and unspecified in 3 (12%) and 0 (0%) cases, respectively. Vascular territory was anterior in 19 (76%) and 10 (100%), posterior in 5 (20%) and 0 (0%), and unspecified in 1 (4%) and 0 (0%) cases. Etiology was large artery disease in 8 (32%) and 6 (60%), cardioembolic in 13 (52%) and 4 (40%), small-vessel in 0 (0%) and 0 (0%), other or undetermined in 4 (16%) and (0 (0%) cases.

### COIN Analysis and Visualization

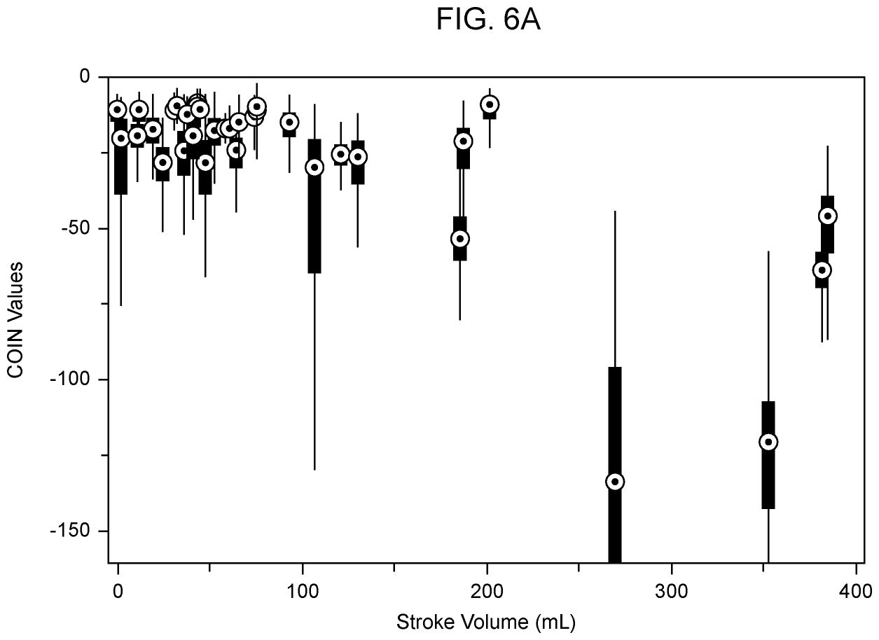

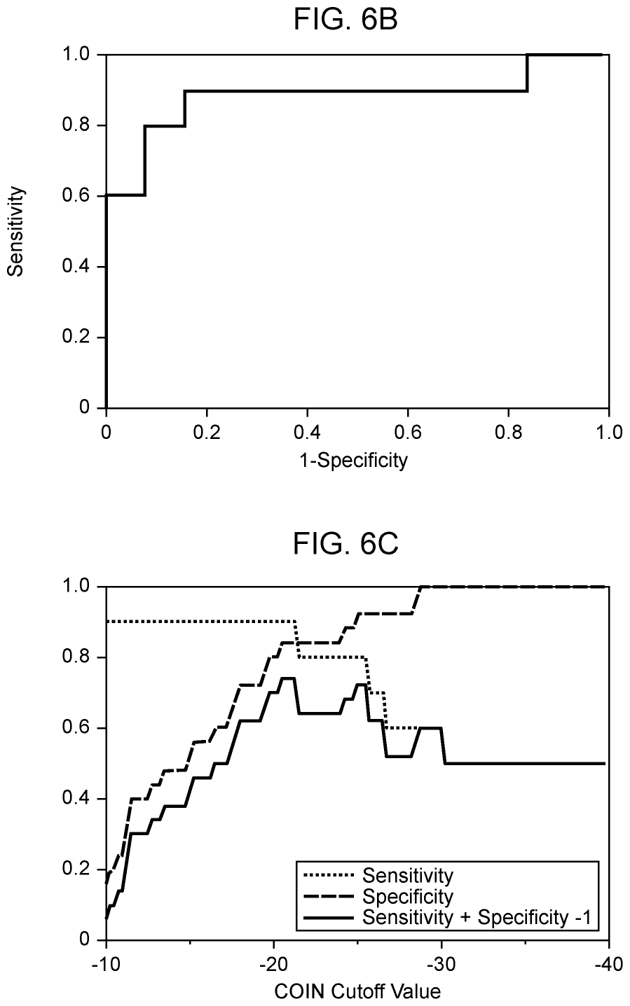

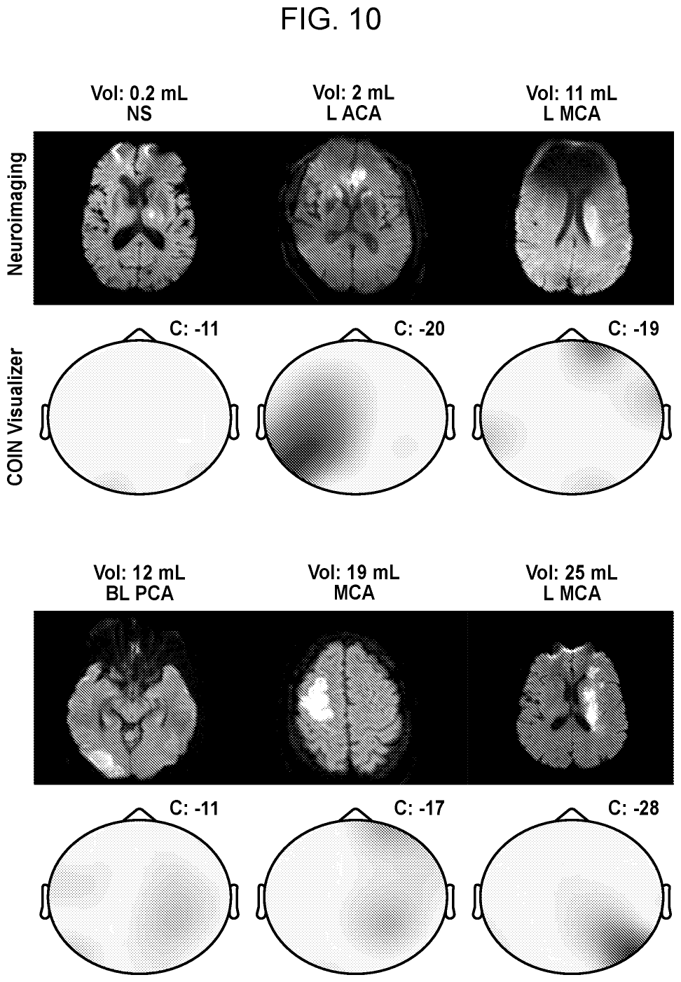

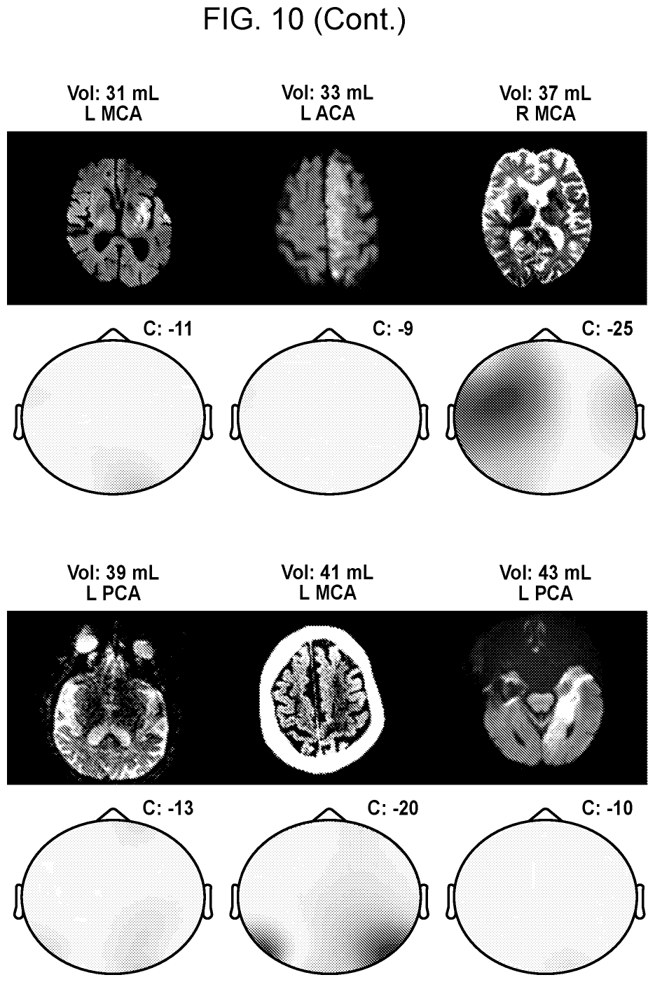

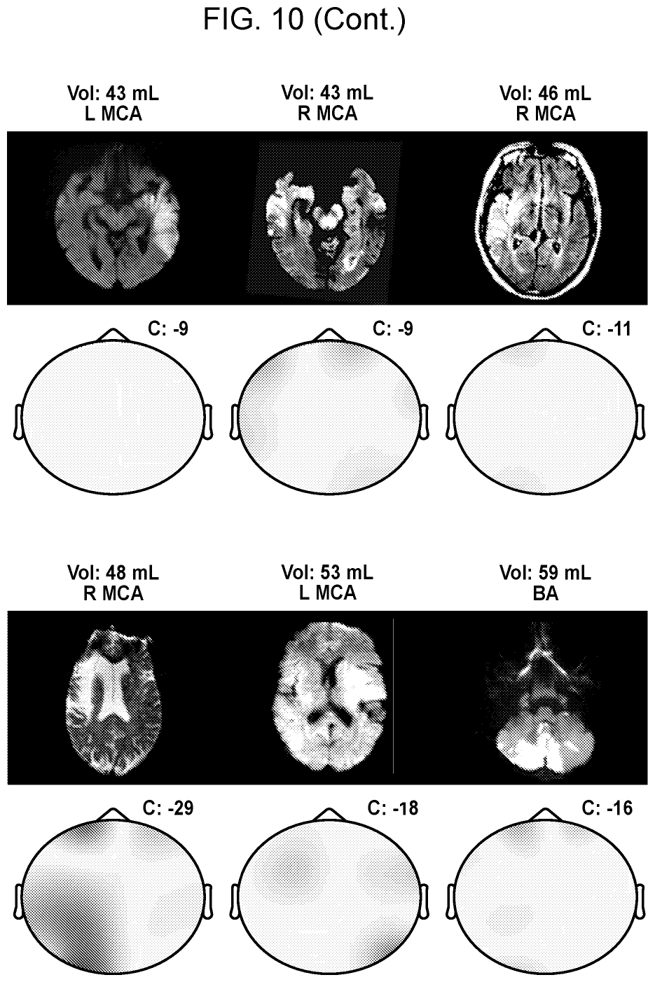

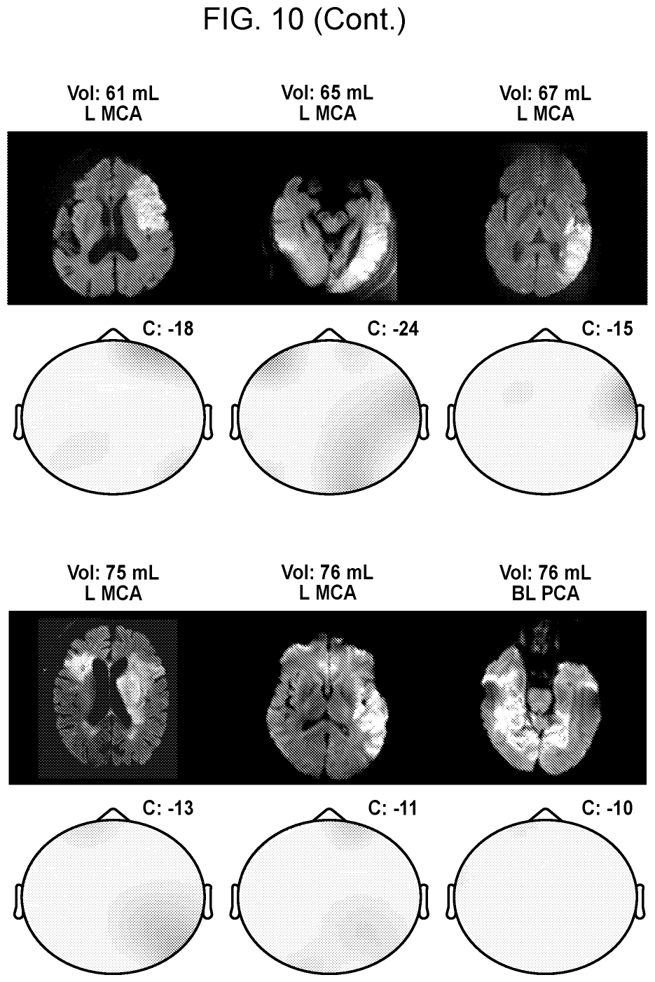

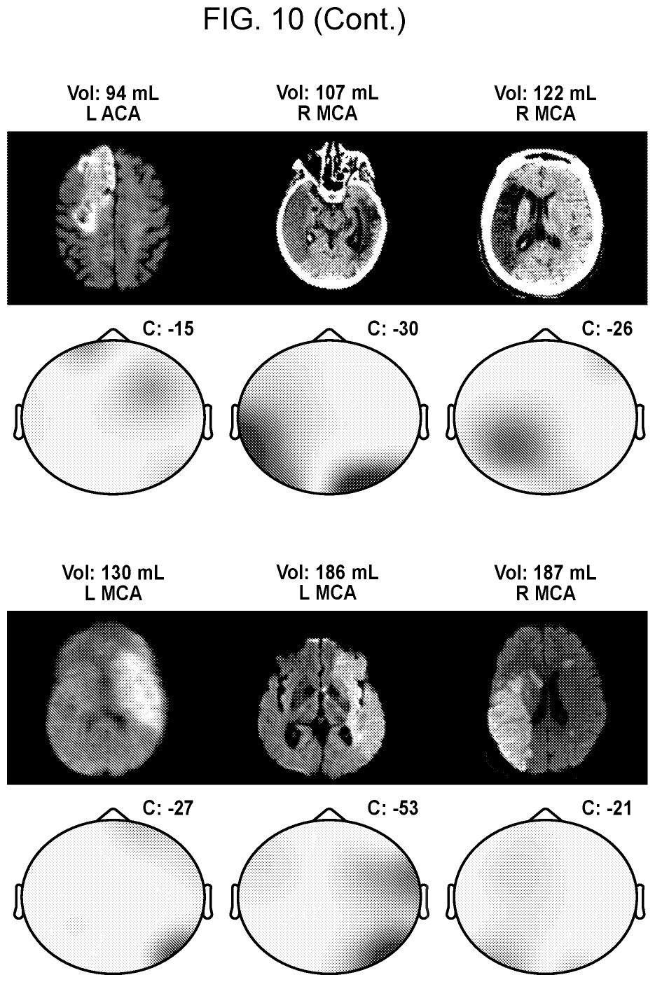

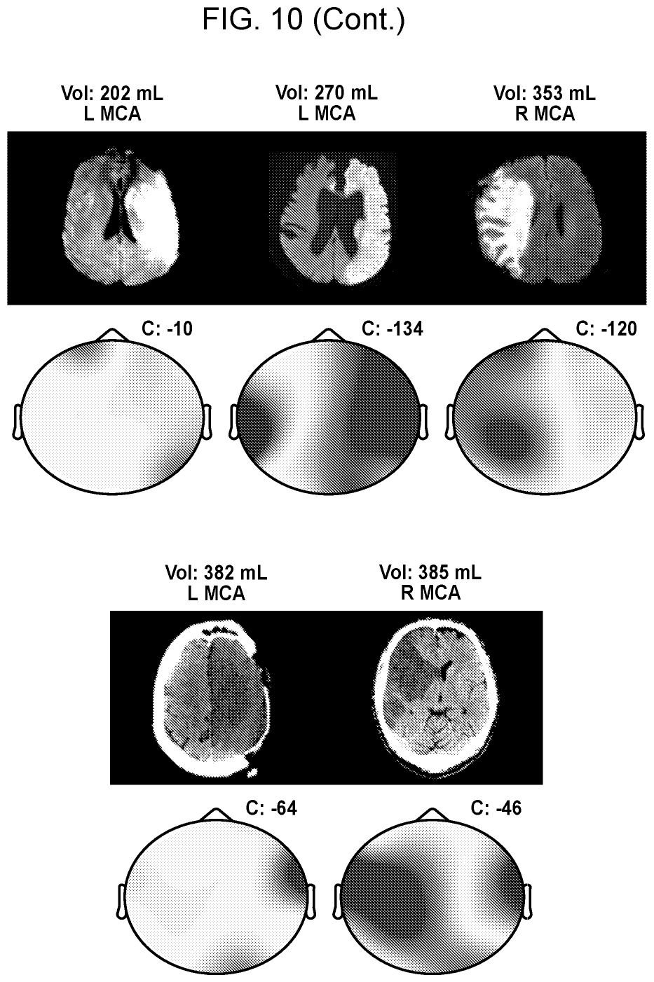

For patients with infarct volume <100 mL and >100 mL, the mean (SD) of median COIN values were −15.7 (1.2) and −53.1 (13) respectively and differed significantly (p=0.0001). Laterality of COIN signal on visualization was concordant with neuroimaging in 11 (44%) and 10 (100%) of cases, discordant in 2 (8%) and 0 (0%) of cases, and indeterminate in 12 (48%) and 0 (0%) of cases. Comparison of representative neuroimages and COIN visualization are shown in FIG. 10. Logistic regression using median COIN values showed an area under the receiver operator characteristic of 0.88 with confidence interval 0.59-0.98. A COIN cutoff value of −20 resulted in the maximal Youden J statistic of 0.74 with corresponding sensitivity of 90%, specificity of 84%, and accuracy of 86%. Specificity of 100% was seen at a COIN cutoff of −28, with corresponding sensitivity of 60%. Median and quartile ranges of COIN values against stroke volume are shown in FIG. 6A, and results of logistic regression are seen in FIGS. 6B and 6C.

### Discussion

This study demonstrates that the quantitative EEG metric COIN provides an intuitive and accurate means of discriminating large (core infarct volume >100 mL) from small strokes (core infarct volume <100 mL) in adults. We found that a cutoff value of −20 could be used to differentiate between large and small strokes, and that COIN values below −28 offers high specificity for the presence of a large vs small volume stroke.

For all but one patient with stroke <100 mL and median COIN <−20, the COIN visualizer was concordant with the reported stroke territory. This may be explained by a mismatch between the infarct core volume and downstream penumbric tissue, suggesting that the patient is either at risk of—or has already suffered—an expansion in stroke territory after imaging occurred.

The findings demonstrate that abnormal COIN values are seen most consistently in anterior circulation strokes. The likely explanation is that surface EEG recordings capture cortical activity from brain tissue that is predominantly supplied by the anterior circulation, which also supplies a larger proportion of cerebral tissue overall. Indeed, strokes were all in the anterior circulation in the group with infarct volume >100 mL. Of the 35 patients analyzed, only 5 had strokes in the posterior circulation of which the lowest COIN value observed was −16. The problem highlighted here is further exemplified in the finding that NIHSS values were similar between the two groups. Small (<100 mL) volume infarcts in highly eloquent regions such as the brainstem or internal capsule may manifest severe neurological dysfunction reflected in high NIHSS value.

### Conclusion

COIN can differentiate large (core volume >100 mL) from small ischemic strokes with good accuracy and high specificity. Prospective implementation and evaluation are required to determine utility of COIN as an aid to stroke diagnosis in patients with a limited exam.

### Example 3: Real-Time Recognition of Stroke

A comatose patient on life support with a high risk of bleeding was monitored for the possibility of seizures as an explanation for altered mental status. A brain scan using magnetic resonance brain imaging prior to initiating EEG showed no evidence of brain abnormality. The patient required mechanical ventilation and sedation.

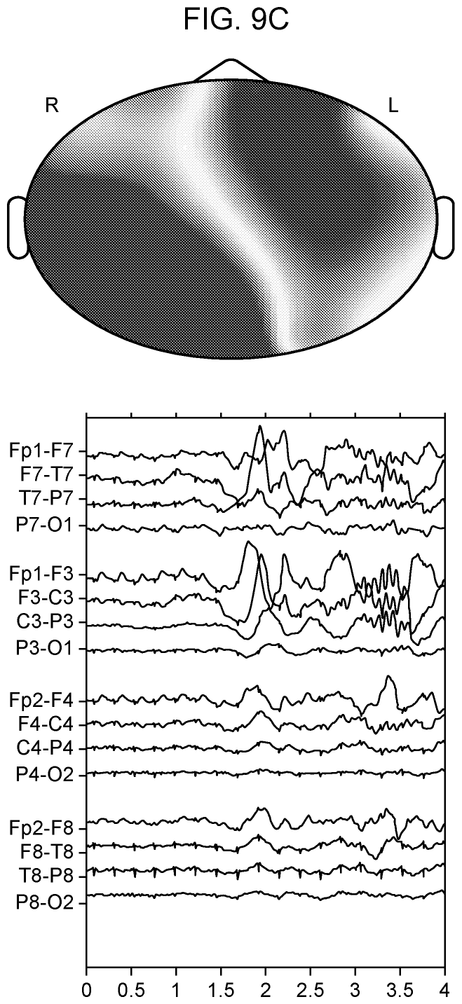

As shown in FIGS. 9A-C, EEG data was recorded and processed to give topographical brain maps. The colors of the brain maps correspond to the “COIN values”, which are also described as m(j) values.

FIG. 9A shows the results of the monitoring while the patient was in a baseline state. The horizontal axis had a time scale of 0 seconds to 4 seconds. The vertical axis shows 16 different channels that were monitored. An epileptologist reviewing the EEG data itself did not note any focal EEG abnormalities. The brain map was mostly white, indicating m(j) values that were close to zero, and therefore suggesting a brain state without focal abnormality.

However, the brain map of FIG. 9B shows a dark blue color in the right posterior quadrant, which indicates focal attenuation. Notably, the right brain is denoted by the letter “R” and is actually shown on the left side of the figure. Thus, the analysis of the EEG data suggested the possibility of a stroke occurring in the right posterior quadrant. The pattern of FIG. 9B persisted for 8 hours. An epileptologist did not note any focal EEG abnormalities during the FIG. 9B time, e.g. because the raw EEG data of FIG. 9B looks similar to the raw EEG data of FIG. 9A.

After about 8 hours, there was a sudden change in the EEG data, as shown in FIG. 9C. The brain map shows a dark blue region on the right posterior quadrant of the brain, and a dark red region was located in the forward left brain. This analysis shows a very high probability of stroke. After the sudden change from the FIG. 9B pattern to the FIG. 9C pattern, an epileptologist noted focal EEG abnormalities. Specifically, the epileptologist noted that the EEG recordings were very flat in FIG. 9C, whereas the EEG recordings had large amplitude in both FIG. 9A and FIG. 9B.

It was determined by computed tomography that the patient had experienced a large intracranial hemorrhage in the right posterior quadrant of the brain.

As such, the interpretation of the EEG data by calculation of COIN values was able to detect abnormalities and a high risk of stroke approximately 8 hours before an epileptologist detected any abnormality in the EEG data itself.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

What is claimed is:

1. A method of assessing whether a subject had a stroke and treating the subject accordingly, the method comprising:
(a) recording electroencephalography (EEG) data from electrodes positioned at scalp locations on the head of the subject;
(b) determining relative electrical activities from the EEG data, wherein the determining comprises:
determining the relative electrical activity at each scalp location compared to electrical activity at all scalp locations; and
determining the relative electrical activity at each scalp location compared to electrical activity at its corresponding contralateral scalp location;
(c) determining a high probability of stroke based on the determined relative electrical activities; and
(d) treating the subject for a high probability of stroke.

2. The method of claim 1, wherein electrodes are positioned at four or more scalp locations.

3. The method of any one of claims 1-2, wherein treating the subject for a high probability of stroke comprises performing an additional stroke detection measurement.

4. The method of claim 3, wherein the additional stroke detection measurement is a brain magnetic resonance image (MRI) or head computed tomography (CT) scan.

5. The method of any one of claims 1-4, wherein treating the subject for a high probability of stroke comprises medical or surgical intervention to reverse or minimize the effect of stroke.

6. The method of any one of claims 1-5, wherein treating the subject for a high probability of stroke comprises transporting the subject to a medical facility, notifying the medical facility of a high probability of stroke, or a combination thereof.

7. The method of any one of claims 1-6, wherein the subject had an elevated risk of stroke before the recording.

8. The method of claim 7, wherein the elevated risk of stroke comprises a risk selected from the group consisting of: altered mental status, loss of motor function, loss of sense of touch in a body part, dizziness, headache, and difficulty speaking.

9. The method of any one of claims 7-8, wherein the elevated risk of stroke comprises a risk selected from the group consisting of: head trauma, hematoma, and subarachnoid hemorrhage.

10. The method of any one of claims 7-9, wherein the elevated risk of stroke comprises a risk selected from the group consisting of: currently receiving surgery and receiving surgery within the past 30 days.

11. The method of any one of claims 1-10, wherein the subject is unconscious.

12. The method of any one of claims 1-11, wherein determining the relative electrical activities from the EEG data comprises:
generating matrix A from the EEG data, wherein matrix A comprises elements a(t, j), wherein a(t, j) refers to the amplitude of electrical activity at time t and scalp location j;
generating matrix B from matrix A, wherein matrix B comprises elements a(f, j), wherein a(f, j) refers to the power of electrical activity at frequency f and scalp location j;
generating referential matrix R comprising elements r(f, j), wherein r(f, j) refers to the relative power of electrical activity at frequency f and scalp location j compared to the power of electrical activity at frequency f at all scalp locations;
generating symmetry matrix S comprising elements s(f, j), wherein s(f, j) refers to the relative power of electrical activity at frequency f and scalp location j compared to the power of electrical activity at frequency f and scalp location j* that is contralateral to scalp location j.

13. The method of claim 12, wherein each r(f, j) is generated by comparing a(f, j) to the mean of a(f) values from all scalp locations j.

14. The method of claim 13, wherein each r(f, j) is described by the equation:
$$r ( f , j ) = {log}_{2} ( \frac{a ( f , j )}{\frac{1}{M} {\sum }_{j = 1}^{M} a ( f , j )} )$$
wherein M is the total number of scalp locations j.

15. The method of any one of claims 12-14, wherein generating each s(f, j) comprises dividing a(f, j) by a(f, j*), wherein j* is a scalp location that is contralateral to scalp location j.

16. The method of claim 15, wherein each s(f, j) is described by the equation:
$$s ( f , j ) = {log}_{2} ( \frac{a ( f , j )}{a ( f , {j}^{⋆} )} ) .$$

17. The method of any one of claims 12-16, wherein determining a high probability of stroke comprises:
generating gating matrix G comprising elements g(f, j) by combining matrix R with matrix S;
generating map matrix M comprising elements m(j), wherein m(j) is generated by averaging g values for the same scalp location j over different frequencies f;
determining a high probability of stroke based on map matrix M.

18. The method of claim 17, wherein generating gating matrix G comprises calculating each g(f, j) according to the equation:
$$g ( f , j ) = {r ( f , j )}^{2} \cdot s ( f , j ) .$$

19. The method of any one of claims 17-18, wherein generating gating matrix G further comprises setting each g(f, j) to zero if its corresponding r(f, j) has the opposite sign of its corresponding s(f, j).

20. The method of any one of claims 17-19, wherein a high probability of stroke is determined when one or more m(j) value is within a predetermined threshold range.

21. The method of claim 20, wherein the predetermined threshold range of one or more m(j) values is less than −15.

22. The method of any one of claims 17-19, wherein a high probability of stroke is determined when the sum of all negative m(j) values is within the predetermined threshold range.

23. The method of claim 20, wherein the predetermined threshold range of the sum of all negative m(j) values is less than −15.

24. The method of any one of claims 17-23, further comprising estimating the location of the stroke by identifying the scalp locations with the most negative m(j) values.

25. The method of any one of claims 1-24, wherein the probability of stroke is the probability of an ischemic stroke.

26. The method of any one of claims 1-24, wherein the probability of stroke is the probability of hemorrhagic stroke.

27. The method of any one of claims 1-26, further comprising determining the size of the stroke based on the relative electrical activities.

28. The method of claim 27, wherein the size of the stroke is determined to be 100 ml or more based on an m(j) value of −20 or less, and the size of the stroke is determined to be less than 100 ml based on an m(j) value of greater than −20.

29. The method of any one of claims 1-25, further comprising:
repeating the recording of the EEG data during a second time period;
determining the relative electrical activities from the EEG data from one or more additional time periods;
generating a combined relative electrical activity by combining the relative electrical activity from the EEG data from the first time period and the relative electrical activity from the EEG data from the first time period; and
determining a high probability of stroke based on the combined relative electrical activities.

30. A controller for assessing whether a subject had a stroke and communicating the results of the assessment, wherein the controller is configured to:
a) obtain electroencephalography (EEG) data from electrodes positioned at scalp locations on the head of the subject;
b) determine relative electrical activities from the EEG data, wherein the calculating comprises:
determining the relative electrical activity at each scalp location compared to electrical activity at all scalp locations; and
determining the relative electrical activity at each scalp location compared to electrical activity at its corresponding contralateral scalp location;
c) determine the probability that the subject experienced a stroke near one or more scalp locations based on the relative electrical activities; and
d) electronically instruct a communication device to communicate the determined probabilities.

31. The controller of claim 30, wherein communicating the determined probabilities comprises providing a visual image indicating the one or more determined probabilities.

32. The controller of claim 31, wherein the image comprises a symbol representing a top view or a bottom view of the head of the subject.

33. The controller of claim 32, wherein the image uses different colors to show different relative probabilities that a stroke occurred near different scalp locations.

34. The controller claim 33, wherein the different colors comprise a gradient between three or more colors.

35. The controller of claim 34, wherein the image uses a gradient of colors from blue to white to red in order to indicate a gradient of stroke probability from high to medium to low.

36. The controller of any one of claims 30-35, wherein the controller is further configured to electronically instruct an alert device to provides a visual alert, an auditory alert, or a combination thereof when the determined probability near one or more scalp locations is within a predetermined threshold range.

37. The controller of any one of claims 30-36, wherein determining the relative electrical activities from the EEG data comprises:
generating matrix A from the EEG data, wherein matrix A comprises elements a(t, j), wherein a(t, j) refers to the amplitude of electrical activity at time t and scalp location j;
generating matrix B from matrix A, wherein matrix B comprises elements a(f, j), wherein a(f, j) refers to the power of electrical activity at frequency f and scalp location j;
generating referential matrix R comprising elements r(f, j), wherein r(f, j) refers to the relative power of electrical activity at frequency f and scalp location j compared to the power of electrical activity at frequency f at all scalp locations; and
generating symmetry matrix S comprising elements s(f, j), wherein s(f, j) refers to the relative power of electrical activity at frequency f and scalp location j compared to the power of electrical activity at frequency f and scalp location j* that is contralateral to scalp location j.

38. The controller of claim 37, wherein each r(f, j) is generated by comparing a(f, j) to the mean of a(f) values from all scalp locations j.

39. The controller of claim 38, wherein each r(f, j) is described by the equation:
$$r ( f , j ) = {log}_{2} ( \frac{a ( f , j )}{\frac{1}{M} {\sum }_{j = 1}^{M} a ( f , j )} )$$
wherein M is the total number of scalp locations j.

40. The controller of any one of claims 37-39, wherein generating each s(f, j) dividing a(f, j) by a(f, j*), wherein j* is a scalp location that is contralateral to scalp location j.

41. The controller of claim 40, wherein each s(f, j) is described by the equation:
$$s ( f , j ) = {log}_{2} ( \frac{a ( f , j )}{a ( f , {j}^{⋆} )} ) .$$

42. The controller of any one of claims 37-41, wherein determining a high probability of stroke comprises:
generating gating matrix G comprising elements g(f, j) by combining matrix R with matrix S;
generating map matrix M comprising elements m(j), wherein m(j) is generated by averaging g values for the same scalp location j over different frequencies f;
determining the probability of stroke based on map matrix M.

43. The controller of claim 36, wherein generating gating matrix G comprises calculating each g(f, j) according to the equation:
$$g ( f , j ) = {r ( f , j )}^{2} \cdot s ( f , j ) .$$

44. The controller of any one of claims 42-43, wherein generating gating matrix G further comprises setting each g(f, j) to zero if its corresponding r(f, j) has the opposite sign of its corresponding s(f, j).

45. The controller of any one of claims 42-44, wherein a high probability of stroke is determined when one or more m(j) value is within a predetermined threshold range.

46. The controller of claim 45, wherein the predetermined threshold range of one or more m(j) values is less than −15.

47. The controller of any one of claims 42-45, wherein a high probability of stroke is determined when C is within a predetermined range, wherein C is the sum of all negative m(j) values.

48. The controller of claim 47, wherein the predetermined threshold range of C is less than −15.

49. The controller of any one of claims 37-48, wherein steps a), b), c) and d) are repeated within 2 minutes or less.

50. The controller of any one of claims 30-49, wherein the probability of stroke is the probability of an ischemic stroke.

51. The controller of any one of claims 30-49, wherein the probability of stroke is the probability of a hemorrhagic stroke.

52. The controller of any one of claims 30-51, wherein the controller is configured to detect stroke with a sensitivity of 70% or more and a specificity of 90% or more.

53. The controller of any one of claims 30-51, wherein the controller is configured to detect stroke with an infarct volume of 5% or more with a sensitivity of 90% or more and a specificity of 90% or more.

54. The controller of any one of claims 30-53, wherein the controller is further configured to determining the size of the stroke based on the relative electrical activities.

55. The controller of claim 54, wherein the size of the stroke is determined to be 100 ml when C is −20 or less, and the size of the stroke is determined to be less than 100 ml when C is greater than −20, wherein C is the sum of all negative m(j) values

56. The controller of any one of any one of claims 54-55, wherein the controller is configured to detect if the size of the stroke is above 100 ml or below 100 ml with a sensitivity of 80% or more and a specificity of 85% or more.

57. The controller of any one of claims 30-56, wherein the controller is further configured to:
repeat the recording of the EEG data during a second time period;
determine the relative electrical activities from the EEG data from one or more additional time periods; and
generate a combined relative electrical activity by combining the relative electrical activity from the EEG data from the first time period and the relative electrical activity from the EEG data from the first time period; and
determine the probability of stroke based on the combined relative electrical activities.

58. A system for assessing whether a subject had a stroke and communicating the results of the assessment, wherein the system comprising:
a controller of any one of claims 30-57; and
one or more of the communication device, an alert device, and the electrodes.

59. A kit for assessing whether a subject had a stroke and communicating the results of the assessment, wherein the system comprising:
a controller of any one of claims 30-57;
packaging containing the controller.

60. The kit of claim 59, further comprising one or more of the communication device, an alert device, and the electrodes contained in the packaging.
